(19) United States
(12) Patent Application Publication
(10) Pub. No.: US 2005/0197327 A1
(43) Pub. Date: Sep. 8, 2005

(54) Compounds, compositions, and methods

(76) Inventors: Gustave Bergnes, Pacifica, CA (US); Dashyant Dhanak, West Chester, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/980,627

(22) Filed: Nov. 2, 2004

Related U.S. Application Data
(60) Provisional application No. 60/517,264, filed on Nov. 3, 2003.

(51) Int. Cl. A61K 31/55; A61K 31/541; A61K 31/5377; A61K 31/513; C07D 417/02; C07D 413/02; C07D 43/02

(52) U.S. Cl. 514/212.08; 514/227.5; 514/235.5; 514/252.14; 514/269; 540/524; 544/60; 544/124; 544/295; 544/310; 514/283

(57) ABSTRACT

Compounds useful for treating cellular proliferative diseases and disorders by modulating the activity of KSP are disclosed.

This application claims the benefit of U.S. Provisional Patent Application No. 60/517,264, filed Nov. 3, 2003, which is incorporated herein by reference for all purposes.

This invention relates to compounds which are inhibitors of the mitotic kinesin KSP and are useful in the treatment of cellular proliferative diseases, for example cancer, hyperplasias, restenosis, cardiac hypertrophy, immune disorders, fungal disorders, and inflammation.

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described (Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426), and a fragment of the KSP gene (TRIP5) has been described (Lee, et al., Mol Endocrinol., 9:243-54 (1995); GenBank accession number L40372). Xenopus KSP homologs (Eg5), as well as Drosophila KLP61 F/KRP1 30 have been reported.

Mitotic kinesins, including KSP, are attractive targets for the discovery and development of novel antimitotic chemotherapeutics. Accordingly, it is an object of the present invention to provide compounds, compositions and methods useful in the inhibition of KSP.

In accordance with the objects outlined above, the present invention provides compounds that can be used to treat cellular proliferative diseases. The compounds are KSP inhibitors, such as human KSP inhibitors. The present invention also provides compositions comprising such compounds, and methods utilizing such compounds or compositions, which can be used to treat cellular proliferative diseases.

In one aspect, the invention relates to at least one chemical entity chosen from compounds of Formula I: 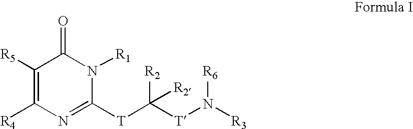
and pharmaceutically acceptable salts, solvates, crystal forms, diastereomers, and prodrugs thereof, wherein:

T and T′ are independently optionally substituted lower alkylene or absent;

R₁ is chosen from hydrogen, optionally substituted alkyl-, optionally substituted aryl-, optionally substituted aralkyl-, optionally substituted heteroaryl-, and optionally substituted heteroaralkyl-;

R₂ and R_{2′} are independently chosen from hydrogen, optionally substituted alkyl-, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;

R₃ is selected from hydrogen, optionally substituted alkyl-, optionally substituted aryl-, optionally substituted aralkyl-, optionally substituted heteroaryl-, optionally substituted heteroaralkyl-, —(CO)R₇, and —SO₂R₇ₐ;

or R₃ taken together with R₆, and the nitrogen to which they are bound, form an optionally substituted 5- to 12-membered nitrogen-containing heterocycle, which optionally incorporates from one to two additional heteroatoms, selected from N, O, and S in the heterocycle ring;

or R₆ taken together with R₂ form an optionally substituted 5- to 12-membered nitrogen-containing heterocycle, which optionally incorporates from one to two additional heteroatoms, selected from N, O, and S in the heterocycle ring;

R₄ and R₅ are independently chosen from hydrogen, optionally substituted alkyl-, optionally substituted alkoxy, acyl, halogen, hydroxy, nitro, cyano, alkylsulfonyl-, alkylsulfanyl-, aminocarbonyl-, optionally substituted amino, optionally substituted aryl-, optionally substituted aralkyl-, optionally substituted heteroaralkyl and optionally substituted heteroaryl-;

R₆ is chosen from hydrogen, optionally substituted alkyl-, optionally substituted aryl-, optionally substituted aralkyl-, optionally substituted heteroaralkyl-, and optionally substituted heterocyclyl-;

R₇ is chosen from hydrogen, optionally substituted alkyl-, optionally substituted aryl-, optionally substituted aralkyl-, optionally substituted heteroaryl-, optionally substituted heteroaralkyl-, R₈O— and R₁₄—NH—;

R₇ₐ is chosen from optionally substituted alkyl-, optionally substituted aryl-, optionally substituted aralkyl-, optionally substituted heteroaryl-, optionally substituted heteroaralkyl-, and R₁₄—NH—;

R₈ is chosen from optionally substituted alkyl-, optionally substituted aryl-, optionally substituted aralkyl-, optionally substituted heteroaryl-, and optionally substituted heteroaralkyl-; and

R₁₄ is chosen from hydrogen, optionally substituted alkyl-, optionally substituted aryl-, optionally substituted aralkyl-, optionally substituted heteroaryl-, and optionally substituted heteroaralkyl- provided that:

at least one the following criteria is met:

T and T′ are not both absent; or

R₆ taken together with R₂ form an optionally substituted 5- to 12-membered nitrogen-containing heterocycle, which optionally incorporates from one to two additional heteroatoms, selected from N, O, and S in the heterocycle ring; or

R₃ taken together with R₆, and the nitrogen to which they are bound, form an optionally substituted 5- to 12-membered nitrogen-containing heterocycle, which optionally incorporates from one to two additional heteroatoms, selected from N, O, and S in the heterocycle ring wherein said heterocycle is not an imidazole or imidazoline ring when T and T′ are both absent.

Accordingly, in some embodiments, R₁, R₂, R_{2′}, and R₃-R₆ are as defined above and one of T and T′ is optionally substituted lower alkylene with the other being absent. In some embodiments, R₁, R₂, R_{2′}, and R₃-R₆ are as defined above and both T and T′ are optionally substituted lower alkylene.

In some embodiments, T, T′, R₁, R_{2′}, and R₃-R₅ are as defined above and R₆ taken together with R₂ form an optionally substituted 5- to 12-membered nitrogen-containing heterocycle, which optionally incorporates from one to two additional heteroatoms, selected from N, O, and S in the heterocycle ring.

In some embodiments, T, T′, R₁, R₂, R_{2′}, R₄ and R₅ are as defined above and R₃ taken together with R₆, and the nitrogen to which they are bound, form an optionally substituted 5- to 12-membered nitrogen-containing heterocycle, which optionally incorporates one or two heteroatoms, selected from N, O, and S in the heterocycle ring, provided that said heterocycle is not an imidazole or imidazoline ring when T and T′ are both absent.

The invention also relates to pharmaceutical compositions comprising: a therapeutically effective amount of at least one chemical entity chosen from compounds of Formula I and pharmaceutically acceptable salts, solvates, crystal forms, diastereomers, and prodrugs thereof; and one or more pharmaceutical excipients. In another aspect, the composition further comprises an additional chemotherapeutic agent.

In one aspect, the invention relates to methods for treating cellular proliferative diseases and other disorders that can be treated by inhibiting KSP by.the administration of a therapeutically effective amount of at least one chemical entity chosen from compounds of Formula I and pharmaceutically acceptable salts, solvates, crystal forms, diastereomers, and prodrugs thereof. Such diseases and disorders include cancer, hyperplasia, restenosis, cardiac hypertrophy, immune disorders, fungal disorders and inflammation.

In an additional aspect, the present invention provides methods of screening for compounds that will bind to a KSP kinesin, for example compounds that will displace or compete with the binding of a compound of the invention. The methods comprise combining a labeled compound of the invention, a KSP kinesin, and at least one candidate agent and determining the binding of the candidate agent to the KSP kinesin.

In a further aspect, the invention provides methods of screening for modulators of KSP kinesin activity. The methods comprise combining a compound of the invention, a KSP kinesin, and at least one candidate agent and determining the effect of the candidate agent on the KSP kinesin activity.

As used in the present specification, the following words and phrases are generally intended to have the meanings as set forth below, except to the extent that the context in which they are used indicates otherwise. The following abbreviations and terms have the indicated meanings throughout:

| | |
|---|---|
| Ac = | acetyl |
| BNB = | 4-bromomethyl-3-nitrobenzoic acid |
| Boc = | t-butyloxy carbonyl |
| Bu = | butyl |
| c- = | cyclo |
| CBZ = | carbobenzoxy = benzyloxycarbonyl |
| DBU = | diazabicyclo[5.4.0]undec-7-ene |
| DCM = | dichloromethane = methylene chloride = CH₂Cl₂ |
| DCE = | dichloroethane |
| DEAD = | diethyl azodicarboxylate |
| DIC = | diisopropylcarbodiimide |
| DIEA = | N,N-diisopropylethylamine |
| DMAP = | 4-N,N-dimethylaminopyridine |
| DMF = | N,N-dimethylformamide |
| DMSO = | dimethyl sulfoxide |
| DVB = | 1,4-divinylbenzene |
| EEDQ = | 2-ethoxy-1-ethoxycarbonyl-1,2-dihydroquinoline |
| Et = | ethyl |
| ETOH = | ethanol |
| Fmoc = | 9-fluorenylmethoxycarbonyl |
| GC = | gas chromatography |
| HATU = | O-(7-Azabenzotriazol-1-yl)-1,1,3,3-tetramethyluronium |
| | hexafluorophosphate |
| HMDS = | hexamethyldisilazane |
| HOAc = | acetic acid |
| HOBt = | hydroxybenzotriazole |
| Me = | methyl |
| mesyl = | methanesulfonyl |
| MTBE = | methyl t-butyl ether |
| NMO = | N-methylmorpholine oxide |
| PEG = | polyethylene glycol |
| Ph = | phenyl |
| PhOH = | phenol |
| PfP = | pentafluorophenol |
| PPTS = | pyridinium p-toluenesulfonate |
| Py = | pyridine |
| PyBroP = | bromo-tris-pyrrolidino-phosphonium hexafluorophosphate |
| rt = | room temperature |
| sat'd = | saturated |
| s- = | secondary |
| t- = | tertiary |
| TBDMS = | t-butyldimethylsilyl |
| TES = | triethylsilyl |
| TFA = | trifluoroacetic acid |
| THF = | tetrahydrofuran |
| TMOF = | trimethyl orthoformate |
| TMS = | trimethylsilyl |
| tosyl = | p-toluenesulfonyl |
| Trt = | triphenylmethyl |

The term alkyl refers to linear, branched, and cyclic aliphatic hydrocarbon structures and combinations thereof, which structures may be saturated or unsaturated. In some embodiments, alkyl groups are those of C₂₀ or below. In some embodiments, alkyl groups are those of C₁₃ or below. Alkyl includes alkanyl, alkenyl and alkynyl residues; such as vinyl, allyl, isoprenyl and the like. When an alkyl residue having a specific number of carbons is named, all geometric isomers having that number of carbons are encompassed; thus, for example, butyl refers to n-butyl, sec-butyl, isobutyl and t-butyl; propyl includes n-propyl, isopropyl, and c-propyl.

The term lower-alkyl refers to alkyl groups of from 1 to 5 carbon atoms, such as from 1 to 4 carbon atoms. Examples of lower-alkyl groups include methyl, ethyl, propyl, isopropyl, butyl, s- and t-butyl and the like.

The term cycloalkyl refers to cyclic aliphatic hydrocarbon groups of from 3 to 13 carbon atoms and is a subset of alkyl. Examples of cycloalkyl groups include c-propyl, c-butyl, c-pentyl, norbornyl, adamantyl and the like.

The term cycloalkyl-alkyl- refers to cycloalkyl attached to the parent structure through a non-cyclic alkyl and is another subset of alkyl. Examples of cycloalkyl-alkyl- include cyclohexylmethyl, cyclopropylmethyl, cyclohexylpropyl, and the like.

Alkylene-, alkenylene-, and alkynylene- are subsets of alkyl, including the same residues as alkyl, but having two points of attachment within a chemical structure. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)—). Likewise, examples of alkenylene include ethenylene (—CH═CH—), propenylene (—CH═CH—CH₂—), and cyclohexylpropenylene (—CH═CHCH(C₆H₁₃)—). Examples of alkynylene include ethynylene (—C≡C—) and propynylene (—CH≡CH—CH₂—).

The term alkoxy or alkoxyl refers to an alkyl group, such as including from 1 to 8 carbon atoms, of a straight, branched, or cyclic configuration, or a combination thereof, attached to the parent structure through an oxygen (i.e., the group alkyl-O—). Examples include methoxy-, ethoxy-, propoxy-, isopropoxy-, cyclopropyloxy-, cyclohexyloxy- and the like. Lower-alkoxy refers to alkoxy groups containing one to four carbons.

The term acyl refers to groups of from 1 to 8 carbon atoms of a straight, branched, or cyclic configuration or a combination thereof, attached to the parent structure through a carbonyl functionality. Such groups may be saturated or unsaturated, and aliphatic or aromatic. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like.

The term lower-acyl refers to acyl groups containing one to four carbons.

Amino refers to the group —NH₂. The term substituted amino refers to the group —NHR or —NRR where each R is independently selected from: optionally substituted alkyl-, optionally substituted alkoxy, optionally substituted aminocarbonyl-, optionally substituted aryl-, optionally substituted heteroaryl-, optionally substituted heterocyclyl-, acyl-, alkoxycarbonyl-, sulfanyl-, sulfinyl and sulfonyl-, e.g., diethylamino, methylsulfonylamino, furanyl-oxy-sulfonamino. Substituted amino also includes the groups —NR^{c}COR^{b}, —NR^{c}CO₂R^{a}, and —NR^{c}CONR^{b}R^{c}, where

R^{a} is an optionally substituted C₁-C₆ alkyl-, aryl-, heteroaryl-, aryl-C₁-C₄ alkyl-, or heteroaryl-C₁-C₄ alkyl-group;

R^{b} is H or optionally substituted C₁-C₆ alkyl-, aryl-, heteroaryl-, aryl-C₁-C₄ alkyl-, or heteroaryl-C₁-C₄ alkyl-group; and

R^{c} is hydrogen or C₁-C₄ alkyl-; and

where each optionally substituted R^{b} group is independently unsubstituted or substituted with one or more substituents independently selected from C₁-C₄ alkyl-, aryl-, heteroaryl-, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Antinitotic refers to a drug for inhibiting or preventing mitosis, for example, by causing metaphase arrest. Some antitumour drugs block proliferation and are considered antimitotics.

Aryl refers to a 6-membered aromatic ring; a bicyclic 9 or 10-membered aromatic ring system in which at least one of the rings in the ring system is aromatic; and a tricyclic 12- to 14-membered aromatic ring system in which at least one of the rings in the ring system is aromatic. The aromatic 6- to 14-membered carbocyclic rings include, e.g., phenyl, naphthyl, indanyl, tetralinyl, and fluorenyl.

Heteroaryl refers to

a 5- or 6-membered aromatic heterocyclic ring containing 1-4 heteroatoms selected from O, N, or S;

a bicyclic 9- or 10-membered ring system in which at least one of the rings in the ring system is aromatic and contains 1-4 heteroatoms selected from O, N, or S; and

a tricyclic 12- to 14-membered ring system in which at least one of the rings in the ring system is aromatic and contains 1-4 heteroatoms selected from O, N, or S. The 5- to 10-membered aromatic heterocyclic rings, i.e., heteroaryl groups, include, e.g., imidazolyl, pyridinyl, indolyl, thienyl, benzopyranonyl, thiazolyl, furanyl, benzimidazolyl, quinolinyl, isoquinolinyl, quinoxalinyl, pyrimidinyl, pyrazinyl, tetrazolyl and pyrazolyl.

The term aralkyl refers to a residue in which an aryl moiety is attached to the parent structure via an alkyl residue. Examples include benzyl, phenethyl, phenylvinyl, phenylallyl and the like.

The term heteroaralkyl refers to a residue in which a heteroaryl moiety is attached to the parent structure via an alkyl residue. Examples include furanylmethyl, pyridinylmethyl, pyrimidinylethyl and the like.

Aralkoxy- refers to the group —O-aralkyl. Similarly, heteroaralkoxy- refers to the group —O-heteroaralkyl-; aryloxy- refers to the group —O-aryl-; acyloxy- refers to the group —O-acyl-; heteroaryloxy- refers to the group —O-heteroaryl-; and heterocyclyloxy- refers to the group —O-heterocyclyl (i.e., aralkyl-, heteroaralkyl-, aryl-, acyl-, heterocyclyl-, or heteroaryl is attached to the parent structure through an oxygen).

Aminocarbonyl refers to the group —CONR^{b}R^{c}, where

R^{b} is H or optionally substituted C₁-C₆ alkyl-, aryl-, heteroaryl-, aryl-C₁-C₄ alkyl-, or heteroaryl-C₁-C₄ alkyl-group; and

R^{c} is hydrogen or C₁-C₄ alkyl-; and

where each optionally substituted R^{b} group is independently unsubstituted or substituted with one or more substituents independently selected from C₁-C₄ alkyl-, aryl-, heteroaryl-, aryl-C₁-C₄ alkyl-, heteroaryl-C₁-C₄ alkyl-, C₁-C₄ haloalkyl-, —OC₁-C₄ alkyl-, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). Aminocarbonyl is meant to include carbamoyl-; lower-alkyl carbamoyl-; benzylcarbamoyl-; phenylcarbamoyl-; methoxymethyl-carbamoyl-; and the like.

The term halogen or halo” refers to fluorine (or fluoro), chlorine (or chloro), bromine (or bromo) or iodine (or iodo). Dihaloaryl, dihaloalkyl, trihaloaryl etc. refer to aryl and alkyl substituted with the designated plurality of halogens (here, 2, 2 and 3, respectively), but not necessarily a plurality of the same halogen; thus 4-chloro-3-fluorophenyl is within the scope of dihaloaryl.

Heterocyclyl refers to a cycloalkyl residue in which one to four of the carbons is replaced by a heteroatom such as oxygen, nitrogen or sulfur. Examples include pyrrolidine, tetrahydrofuran, tetrahydro-thiophene, thiazolidine, piperidine, tetrahydro-pyran, tetrahydro-thiopyran, piperazine, morpholine, thiomorpholine and dioxane. Heterocyclyl also includes ring systems including unsaturated bonds, provided the number and placement of unsaturation does not render the group aromatic. Examples include imidazoline, oxazoline, tetrahydroisoquinoline, benzodioxan, benzodioxole and 3,5-dihydrobenzoxazinyl.

A leaving group or atom is any group or atom that will, under the reaction conditions, cleave from the starting material, thus promoting reaction at a specified site. Suitable examples of such groups unless otherwise specified are halogen atoms, mesyloxy, p-nitrobenzensulphonyloxy and tosyloxy groups.

Optional or optionally means that the subsequently described event or circumstance may or may not occur, and that the description includes instances where said event or circumstances occurs and instances in which it does not. For example, “optionally substituted alkyl” includes “alkyl” and “substituted alkyl” as defined herein. It will be understood by those skilled in the art with respect to any group containing one or more substituents that such groups are not intended to introduce any substitution or substitution patterns that are sterically impractical and/or synthetically non-feasible and/or inherently unstable.

Substituted alkoxy refers to alkoxy wherein the alkyl constituent is substituted (i.e., —O-(substituted alkyl)). One suitable substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of about 2-20, such as about 2-10, for example, about 2-5. Another substituted alkoxy group is hydroxyalkoxy or —OCH₂(CH₂)_{y}OH, where y is an integer of about 1-10, such as about 1-4.

Substituted- alkyl-, aryl-, and heteroaryl- refer respectively to alkyl-, aryl-, and heteroaryl wherein one or more (up to five, such as one, two, or three) hydrogen atoms are replaced by a substituent independently selected from: —R¹, —OR^{b}, —O(C₁-C₂ alkyl)O— (as an aryl substituent), —SR^{b}, guanidine, guanidine wherein one or more of the guanidine hydrogens are replaced with a lower-alkyl group, —NR^{b}R^{c}, halogen, cyano, nitro, —COR^{b}, —CO₂R^{b}—CONR^{b}R^{c}, —OCOR^{b}, —OCO₂R^{a}, —OCONR^{b}R^{c}, —NRC^{c}OR^{b}, —NRC^{c}O₂R^{a}, —NR^{c}CONR^{b}R, —CONR^{b}R^{c}, —NRC^{c}OR^{a}, —SOR^{a}, —SO₂R^{a}, —SO₂NR^{b}R^{c}, and —NR^{c}SO₂R^{a},

where R^{a} is an optionally substituted C₁-C₆ alkyl-, aryl-, heteroaryl-, aryl-C₁-C₄ alkyl-, or heteroaryl-C₁-C₄ alkyl-group,

R^{b} is H or optionally substituted C₁-C₆ alkyl-, aryl-, heteroaryl-, aryl-Cl-C₄ alkyl-, or heteroaryl-C₁-C₄ alkyl-group;.

R^{c} is hydrogen or C₁-C₄ alkyl-;

where each optionally substituted R^{a} group and R^{b} group is independently unsubstituted or substituted with one or more substituents independently selected from C₁-C₄ alkyl-, aryl-, heteroaryl-, aryl-C₁-C₄ alkyl-, heteroaryl-C₁-C₄ alkyl-, C₁-C₄ haloalkyl-, —OC₁-C₄ alkyl-, —OC₁-C₄ alkylphenyl-, —C₁-C₄ alkyl-OH, —OC₁-C₄ haloalkyl-, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl-, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl-, —C(O)C₁-C₄ phenyl-, —C(O)C₁-C₄ haloalkyl-, —OC(O)C₁-C₄ alkyl-, —SO₂(C₁-C₄ alkyl), —SO₂NH(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In the compounds of Formula I where T and/or T′ are substituted alkylene, the term “substituted” also refers to alkylene groups where one or more (one or more, such as one, two, or three, for example, one) carbon atoms are replaced by a heteroatom independently selected from O, N or S, such as —CH₂—S—CH₂—.

Sulfanyl refers to the groups: —S-(optionally substituted alkyl), —S-(optionally substituted aryl), —S-(optionally substituted heteroaryl), and —S-(optionally substituted heterocyclyl).

Sulfinyl refers to the groups: —S(O)—H, —S(O)-(optionally substituted alkyl), —S(O)-optionally substituted aryl), —S(O)-(optionally substituted heteroaryl), —S(O)-(optionally substituted heterocyclyl); and —S(O)-(optionally substituted amino).

Sulfonyl refers to the groups: —S(O₂)—H, —S(O₂)-(optionally substituted alkyl), —S(O₂)-optionally substituted aryl), —S(O₂)-(optionally substituted heteroaryl), —S(O₂)-(optionally substituted heterocyclyl), —S(O₂)-(optionally substituted alkoxy), —S(O₂)-optionally substituted aryloxy), 1'S(O₂)-(optionally substituted heteroaryloxy), —S(O₂)-(optionally substituted heterocyclyloxy); and —S(O₂)-(optionally substituted amino).

Pharmaceutically acceptable salts refers to those salts that retain the biological effectiveness of the free compound and that are not biologically undesirable or unsuitable for pharmaceutical use, formed with a suitable acid or base, and includes pharmaceutically acceptable acid addition salts and base addition salts. Pharmaceutically acceptable acid addition salts include those derived from inorganic acids such as hydrochloric acid, hydrobromic acid, sulfuric acid, nitric acid, phosphoric acid and the like, and those derived from organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts include those derived from inorganic bases such as sodium, potassium, lithium, ammonium, calcium, magnesium, iron, zinc, copper, manganese, aluminum salts and the like. In some embodiments, the pharmaceutically acceptable base addition salt is chosen ammonium, potassium, sodium, calcium, and magnesium salts. Base addition salts also include those derived from pharmaceutically acceptable organic non-toxic bases, including salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Protecting group has the meaning conventionally associated with it in organic synthesis, i.e. a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety. For example, a hydroxy protected form is where at least one of the hydroxy groups present in a compound is protected with a hydroxy protecting group. Likewise, amines and other reactive groups may similarly be protected.

Solvate refers to the compound formed by the interaction of a solvent and a compound of Formula I or salt thereof. Suitable solvates of the compounds of the Formula I or a salt thereof are pharmaceutically acceptable solvates including hydrates.

Many of the compounds described herein contain one or more asymmetric centers (e.g. the carbon to which R₂ and R_{2′} are attached where R₂ differs from R_{2′}) and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms and rotational isomers are also intended to be included.

When desired, the R- and S-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

A class of novel compounds, that can be described as pyrimidinone derivatives and that are inhibitors of one or more mitotic kinesins are provided. By inhibiting mitotic kinesins, but not other kinesins (e.g., transport kinesins), specific inhibition of cellular proliferation is accomplished. While not intending to be bound by any theory, the present invention capitalizes on the finding that perturbation of mitotic kinesin function causes malformation or dysfunction of mitotic spindles, frequently resulting in cell cycle arrest and cell death. In some embodiments, the compounds described herein inhibit the mitotic kinesin, KSP, such as human KSP. In some embodiments, the compounds inhibit the mitotic kinesin, KSP, as well as modulating one or more of the human mitotic kinesins selected from HSET (see, U.S. Pat. No. 6,361,993, which is incorporated herein by reference); MCAK (see, U.S. Pat. No. 6,331,424, which is incorporated herein by reference); CENP-E (see, U.S. Pat. No. 6,645,748, which is incorporated herein by reference); Kif4 (see, U.S. Pat. No. 6,440,684, which is incorporated herein by reference); MKLP1 (see, U.S. Pat. No. 6,448,025, which is incorporated herein by reference); Kifl5 (see, U.S. Pat. No. 6,355,466, which is incorporated herein by reference); Kid (see, U.S. Pat. No. 6,387,644, which is incorporated herein by reference); Mpp1, CMKrp, KinI-3 (see, U.S. Pat. No. 6,461,855, which is incorporated herein by reference); Kip3a (see, U.S. Pat. No. 6,680,369, which is incorporated herein by reference); Kip3d (see, U.S. Pat. No. 6,492,151, which is incorporated herein by reference); and RabK6.

The methods of inhibiting a mitotic kinesin comprise contacting a compound of the invention with a kinesin, such as a human kinesin, for example, human KSP or fragments and variants thereof. The inhibition can be of the ATP hydrolysis activity of the KSP kinesin and/or the mitotic spindle formation activity, such that. the mitotic spindles are disrupted. Meiotic spindles may also be disrupted.

The present invention provides inhibitors of mitotic kinesins, such as KSP, for example, human KSP, for the treatment of disorders associated with cell proliferation. The compounds, compositions and methods described herein can differ in their selectivity and are used to treat diseases of cellular proliferation, including, but not limited to cancer, hyperplasias, restenosis, cardiac hypertrophy, immune disorders, fungal disorders and inflammation.

Accordingly, the invention relates to at least one chemical entity chosen from compounds of Formula I: 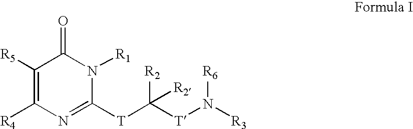
and pharmaceutically acceptable salts, solvates, crystal forms, diastereomers, and prodrugs thereof, wherein:

T and T′ are independently optionally substituted lower alkylene or absent;

R₁ is chosen from hydrogen, optionally substituted alkyl-, optionally substituted aryl-, optionally substituted aralkyl-, optionally substituted heteroaryl-, and optionally substituted heteroaralkyl-;

R₂ and R_{2′} are independently chosen from hydrogen, optionally substituted alkyl-, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;

R₃ is selected from hydrogen, optionally substituted alkyl-, optionally substituted aryl-, optionally substituted aralkyl-, optionally substituted heteroaryl-, optionally substituted heteroaralkyl-, —(CO)R₇, and —SO₂R₇ₐ;

or R₃ taken together with R₆, and the nitrogen to which they are bound, form an optionally substituted 5- to 12-membered nitrogen-containing heterocycle, which optionally incorporates from one to two additional heteroatoms, selected from N, O, and S in the heterocycle ring;

or R₆ taken together with R₂ form an optionally substituted 5- to 12-membered nitrogen-containing heterocycle, which optionally incorporates from one to two additional heteroatoms, selected from N, O, and S in the heterocycle ring;

R₄ and R₅ are independently chosen from hydrogen, optionally substituted alkyl-, optionally substituted alkoxy, acyl, halogen, hydroxy, nitro, cyano, alkylsulfonyl-, alkylsulfanyl-, aminocarbonyl-, optionally substituted amino, optionally substituted aryl-, optionally substituted aralkyl-, optionally substituted heteroaralkyl and optionally substituted heteroaryl-;

R₆ is chosen from hydrogen, optionally substituted alkyl-, optionally substituted aryl-, optionally substituted aralkyl-, optionally substituted heteroaralkyl-, and optionally substituted heterocyclyl-;

R₇ is chosen from hydrogen, optionally substituted alkyl-, optionally substituted aryl-, optionally substituted aralkyl-, optionally substituted heteroaryl-, optionally substituted heteroaralkyl-, R₈O— and R₁₄—NH—;

R₇ₐ is chosen from optionally substituted alkyl-, optionally substituted aryl-, optionally substituted aralkyl-, optionally substituted heteroaryl-, optionally substituted heteroaralkyl-, and R₁₄—NH—;

R₈ is chosen from optionally substituted alkyl-, optionally substituted aryl-, optionally substituted aralkyl-, optionally substituted heteroaryl-, and optionally substituted heteroaralkyl-; and

R₁₄ is chosen from hydrogen, optionally substituted alkyl-, optionally substituted aryl-, optionally substituted aralkyl-, optionally substituted heteroaryl-, and optionally substituted heteroaralkyl-;

provided that:

at least one the following criteria is met:

T and T′ are not both absent; or

R₆ taken together with R₂ form an optionally substituted 5- to 12-membered nitrogen-containing heterocycle, which optionally incorporates from one to two additional heteroatoms, selected from N, O, and S in the heterocycle ring; or

R₃ taken together with R₆, and the nitrogen to which they are bound, form an optionally substituted 5- to 12-membered nitrogen-containing heterocycle, which optionally incorporates from one to two additional heteroatoms, selected from N, O, and S in the heterocycle ring wherein said heterocycle is not an imidazole or imidazoline ring when T and T′ are both absent. In some embodiments, the stereogenic center to which R₂ and R_{2′} are attached is of the R configuration.

The compounds of Formula I can be named and numbered in the manner (e.g., using AutoNom version 2.1 in ISIS-DRAW or ChemDraw) described below. For example, the compound: 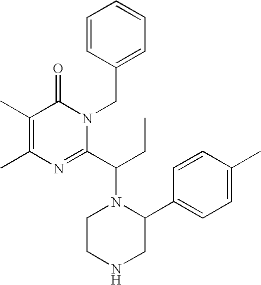
i.e., the compound according to Formula I where T and T′ are absent, R₁ is benzyl-, R₂ is ethyl, R_{2′} is hydrogen; R₃ taken together with R₆, and the nitrogen to which they are bound, is 2-(p-tolyl-piperazin-1-yl); R₄ is methyl-; and R₅ is methyl can be named 3-benzyl-5,6-dimethyl-2-[1-(2-p-tolyl-piperazin-1-yl)-propyl]-3H-pyrimidin-4-one.

Likewise, the compound 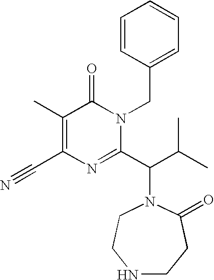
i.e., the compound according to Formula I where T and T′ are absent, R₁ is benzyl-, R₂ is isopropyl, R_{2′} is hydrogen; R₃ together with R₆ form a hexahydro-5H-1,4-diazepin-5-one, R₄ is cyano, and R₅ is methyl can be named 1-benzyl-5-methyl-2-[2-methyl-1-(7-oxo-[1,4]diazepan-1-yl)-propyl]-6-oxo-1,6-dihydro-pyrimidine-4-carbonitrile.

The compounds of Formula I can be prepared by following the procedures described with reference to the Reaction Schemes below.

Unless specified otherwise, the terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

In general, esters of carboxylic acids may be prepared by conventional esterification procedures, for example alkyl esters may be prepared by treating the required activated carboxylic acid with the appropriate alkanol, generally under acidic conditions. Likewise, amides may be prepared using conventional amidation procedures, for example amides may be prepared by treating an activated carboxylic acid with the appropriate amine. Alternatively, a lower-alkyl ester such as a methyl ester of the acid may be treated with an amine to provide the required amide, optionally in presence of trimethylaluminium following the procedure described in Tetrahedron Lett. 48, 4171-4173, (1977). Carboxyl groups may be protected as alkyl esters, for example methyl esters, which esters may be prepared and removed using conventional procedures, one convenient method for converting carbomethoxy to carboxyl is to use aqueous lithium hydroxide.

The salts and solvates of the compounds mentioned herein may as required be produced by methods conventional in the art. For example, if an inventive compound is an acid, a desired base addition salt can be prepared by treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; such as ethylenediamine, and cyclic amines, such as cyclohexylamine, piperidine, morpholine, and piperazine; as well as inorganic salts derived from sodium, calcium, potassium, magnesium, manganese, iron, copper, zinc, aluminum, and lithium.

If a compound is a base, a desired acid addition salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

The compounds of Formula I can be prepared by following the procedures with reference to the Reaction Schemes below. The optionally substituted β-keto amides of Formula 101 and the other reactants are commercially available, e.g., from Aldrich Chemical Company, Milwaukee, Wis. or may be readily prepared by those skilled in the art using commonly employed synthetic methodology. See, for example, PCT WO 03/39460, WO 03/49678, WO 03/50122, WO 03/49527, WO 03/49679, WO 03/50064, US 2004-0077662, US 2004-0077662, and PCT/US03/13627, each of which is incorporated herein by reference for all purposes. 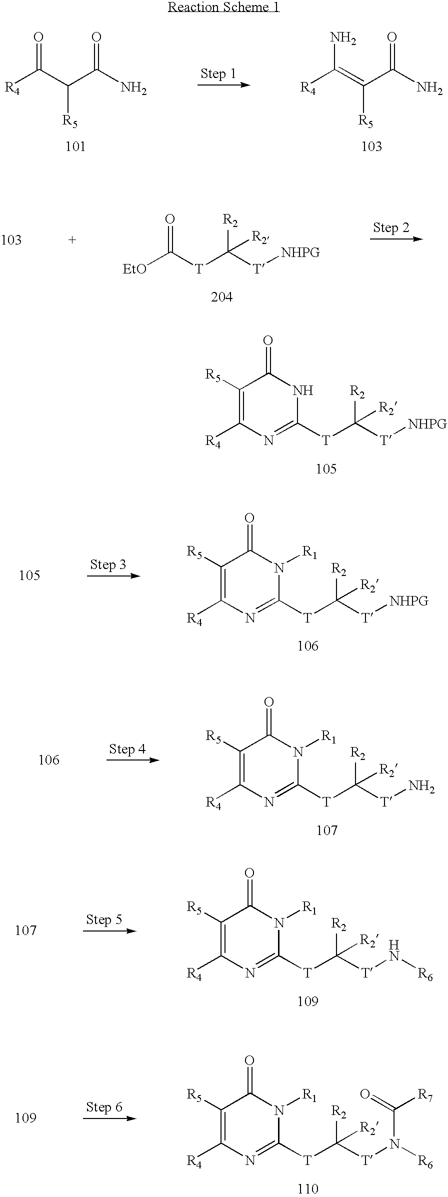
Preparation of Formula 103

Referring to Reaction Scheme 1, Step 1, a mixture of an optionally substituted acetoacetamide (the compound of Formula 101) or an acetoacetate ester in an inert organic solvent (such as xylenes) is added to a flask equipped with a dry-ice reflux condenser. The resulting mixture is heated to reflux and purged continuously with gaseous ammonia for about 3 hours, and then cooled to room temperature. The reaction mixture is filtered and the filtrate is concentrated under reduced pressure. The optionally substituted beta-aminocrotonamide (the compound of Formula 103) is isolated and purified.

Preparation of Formula 105

Referring to Reaction Scheme 1, Step 2, freshly generated sodium ethoxide is added to a mixture of a compound of Formula 103 and a slight excess (such as about 1.1 equivalents) of a suitably protected amino acid ester (a compound of Formula 104, for example, wherein protecting group, PG, is Boc) in ethanol. The resulting solution is heated at reflux for several hours. The product, a pyrimidinone of Formula 105, is isolated and purified.

Preparation of Formula 106

Referring to Reaction Scheme 1, Step 3, to a solution of a pyrimidinone of Formula 105 in a polar, aprotic solvent such as dioxane is added an excess (such as about 1.2 equivalents) of lithium hydride, while maintaining room temperature. The resulting suspension is stirred for about 15 minutes, followed by addition of a slight excess (such as about 1.1 equivalents) of a compound having the structure R₁—X wherein X is a leaving group, such as a tosylate and R₁ is as defined above. The reaction mixture is heated at reflux for about 20-24 hours. The product, a pyrimidinone of Formula 106, is isolated and purified.

Preparation of Formula 107

Referring to Reaction Scheme 1, Step 4, the amino protecting group of a compound of Formula 106 is removed. For example, to a solution of a pyrimidinone of Formula 106 wherein the amino protecting group, PG, is Boc in a polar, aprotic solvent such as dichloromethane is added trifluoroacetic acid, while maintaining the temperature at about 0° C. The resulting solution is then stirred at room temperature for one hour and concentrated in vacuo. The product, a compound of Formula 107, is isolated and used in the next step without firther purification. One of skill in the art will readily appreciate that the removal of other protecting groups can be accomplished using conditions known in the art. See, e.g., Greene, et al. supra.

Preparation of Formula 109

Referring to Reaction Scheme 1, Step 5, to a solution of a pyrimidinone of Formula 107 is added successively a slight excess (such as about 1.2 equivalents) of an aldehyde comprising R_{6′} (i.e., a compound having the formula R_{6′}CHO where R_{6′}CH₂— is equivalent to R₆ and R₆ is as described above or is a protected precursor to such a substituent, e.g., (3-oxo-propyl)-carbamic acid tert-butyl ester) and a reducing agent such as sodium triacetoxyborohydride. The resulting mixture is stirred for several hours. The product, a pyrimidinone of Formula 109, is isolated and purified.

Preparation of Formula 110

Referring to Reaction Scheme 1, Step 6, to a solution of a pyrimidinone of Formula 109 and an amine base such as diisopropylethylamine in a polar, aprotic solvent such as dichloromethane is added an R₇ acyl chloride (such as Cl—C(O)—R₇ where R₇ is as described above). The resulting solution is stirred under nitrogen at room temperature for several hours. The product, a pyrimidinone of Formula 110, is isolated and purified.

Optionally, any protecting groups on a compound of Formula 110 are then removed. For example, if R₆ comprises a protected amine wherein the protecting group is a Boc group, the Boc may be removed by treating a solution of a pyrimidinone of Formula 110 in a polar, aprotic solvent such as dichloromethane is added trifluoroacetic acid, while maintaining the reaction at about room temperature. The reaction is monitored, e.g., by TLC. Upon completion, the free amine is isolated and purified.

Preparation of Optically Active Compounds of Formula 107

In certain compounds of the invention, a particular stereo configuration (such as the (R) isomer) may be preferred at the stereogenic center to which R₂ is attached. The optically active compound can be prepared by methods known in the art. For example, an amine of Formula 107 is dissolved in an inert organic solvent (such as IPA) and warmed to 60° C. In a separate vessel, a resolving agent (such as dibenzoyl-D-tartaric acid) is dissolved, and then quickly added (with agitation) to the warm amine solution. The reaction mixture is left to crystallize by cooling to room temperature over 16 hours under continuing agitation. The desired isomer, e.g., the (R) isomer, is isolated and purified.

For the sake of brevity in the remaining description of the synthesis of compounds of Formula I, it should be understood that either single isomer or a mixture of isomers may be employed to give the corresponding product. 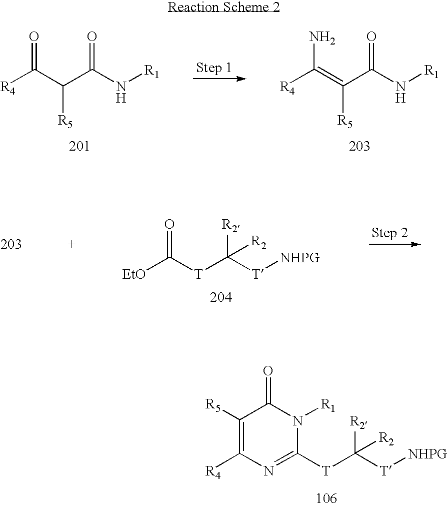
Preparation of Formula 203

Referring to Reaction Scheme 2, Step 1, a mixture of an optionally substituted beta-ketoamide of Formula 201 in an inert organic solvent (such as xylenes) is added to a flask equipped with a dry-ice reflux condenser. The resulting mixture is heated to reflux and purged continuously with gaseous ammonia for about 5 hours, and then cooled to room temperature. The reaction mixture is filtered and the filtrate is concentrated under reduced pressure. The product, an optionally substituted compound of Formula 203, is isolated and used in the next step without further purification.

Preparation of Formula 106

Referring to Reaction Scheme 2, Step 2, freshly generated sodium ethoxide is added to a mixture of a compound of Formula 203 and a slight excess (such as about 1.1 equivalents) of a suitably protected amino acid ester (a compound of Formula 204, such as a compound of Formula 204 wherein PG is Boc) in ethanol. The resulting solution is heated at reflux for several hours. The product, a pyrimidinone of Formula 106, is isolated and purified.

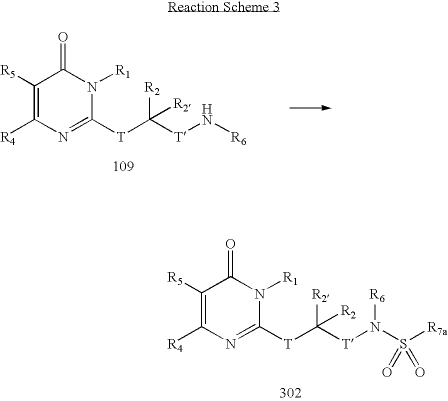

Referring to Reaction Scheme 3, to a solution of a pyrimidinone of Formula 109 and an amine base such as diisopropylethylamine in a polar, aprotic solvent such as dichloromethane is added a compound having the formula Cl—S(O)₂—R₇a or O—(S(O)₂—R₇ₐ)₂ where R₇ₐ is as described above. The resulting solution is stirred under nitrogen at room temperature for several hours. The product, a pyrimidinone of Formula 302, is isolated and purified.

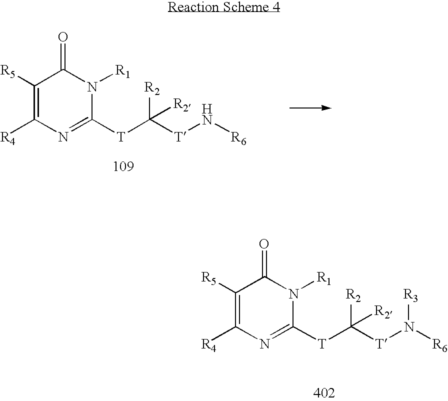

Referring to Reaction Scheme 4, to a solution of a pyrimidinone of Formula 109 and an amine base such as diisopropylethylamine in a polar, aprotic solvent such as dichloromethane is added a compound having the formula X—R₃ where R₇ is as described above and X is a leaving group (such as a halide). The resulting solution is stirred under nitrogen at room temperature or with heat for several hours. The product, a pyrimidinone of Formula 402, is isolated and purified. 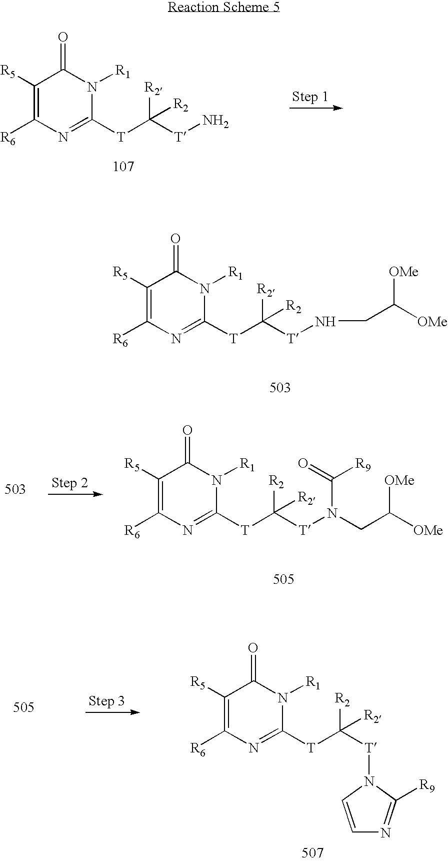
Preparation of Formula 503

Referring to Reaction Scheme 5, Step 1, to an optionally substituted compound of Formula 107 dissolved in a polar, aprotic solvent (such as DMF) in the presence of a base (such as potassium carbonate) is added one equivalent of an optionally substituted suitably protected aldehyde wherein such aldehyde further comprises a leaving group, such as, a halide. The solution is heated at reflux, monitoring completion of the reaction (e.g., by TLC). The reaction mixture is cooled and the corresponding, optionally substituted pyrimidinone of Formula 503 is isolated and purified.

Preparation of Formula 505

Referring to Reaction Scheme 5, Step 2, to an optionally substituted compound of Formula 503 in an inert solvent (such as dichloromethane) in the presence of about 1.5 molar equivalents of an amine base (such as triethylamine) is added about 1.5 molar equivalents of an R₉ acid chloride, such as, Cl—C(O)—R₉, where R₉ is as described herein. The reaction takes place, with stirring, at room temperature over a period of 4 to 24 hours. Completion is monitored, e.g., by TLC. The corresponding compound of Formula 505 is isolated and purified.

Preparation of Formula 507

Referring to Reaction Scheme 5, Step 3, a solution of a compound of Formula 505 and an excess of ammonium acetate in acetic acid is heated at reflux for 1-4 hours. Completion is monitored, e.g., by TLC. The corresponding compound of Formula 507 is isolated and purified. 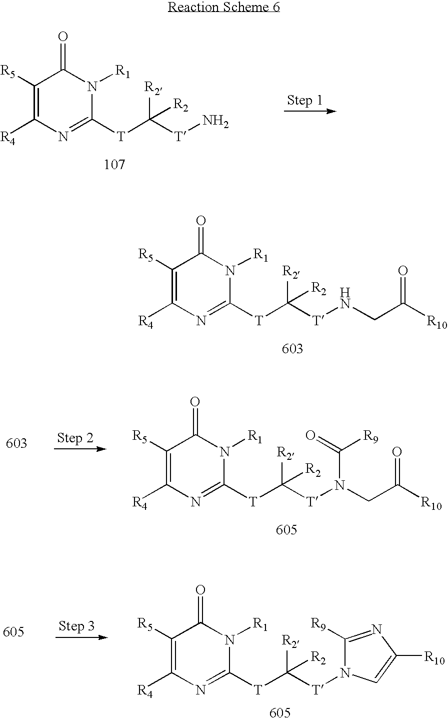
Preparation of Formula 603

Referring to Reaction Scheme 6, Step 1, a suspension of a compound of Formula 107, an alpha-haloketone reagent of the Formula R₁₀(CO)CH₂X wherein X is a halide, and about an equivalent of a base, such as potassium carbonate in a polar, aprotic solvent such as DMF is stirred at room temperature. The reaction is diluted with water and the resulting solid, a compound of Formula 603, is used in the subsequent step without further purification.

Preparation of Formula 605

Referring to Reaction Scheme 6, Step 2, a solution of the compound of Formula 603, about an equivalent of an amine base, such as triethylamine and about an equivalent of an acid chloride (such as a compound of Formula R₉—COCl) in an organic solvent such as methylene chloride is stirred at room temperature for several hours. Completion is monitored, e.g., by TLC. The corresponding compound of Formula 605 is isolated and purified.

Preparation of Formula 607

Referring to Reaction Scheme 6, Step 3, a solution of a compound of Formula 605 and an excess of ammonium acetate in acetic acid is heated at reflux using a Dean-Stark trap and condenser. Completion is monitored, e.g., by TLC. The corresponding compound of Formula 607 is isolated and purified.

Optionally, if a compound of Formula 607 is protected as a phthalimide, a solution of a compound of Formula 607 and an excess of anhydrous hydrazine in a polar, protic solvent such as ethanol is heated at reflux. The reaction is cooled to about 5° C. and any precipitate is filtered off. The filtrate is concentrated in vacuo and purified to yield the free amine. 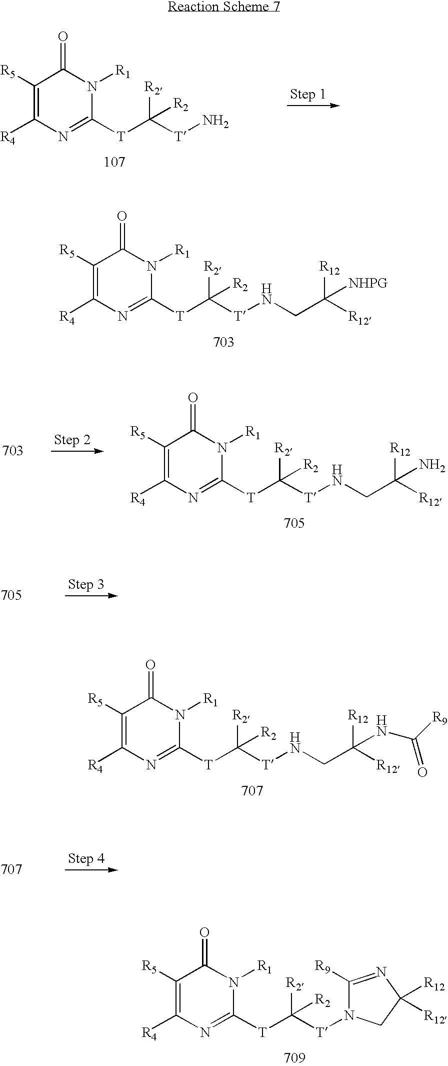
Preparation of Formula 703

Referring to Reaction Scheme 7, Step 1, to a solution of a compound of Formula 107 and an equivalent of a suitably protected aldehyde (Seki et. al. Chem. Pharm. Bull. 1996, 44, 2061) in dichloromethane is added a slight excess of a reducing agent, such as sodium triacetoxyborohydride. The resultant cloudy mixture is maintained at ambient temperature. Completion is monitored, e.g., by TLC. The corresponding compound of Formula 703 is isolated and used in the subsequent step without purification.

Preparation of Formula 705

Referring to Reaction Scheme 4, Step 2, to a solution of a compound of Formula 703 in a polar, aprotic solvent such as dichloromethane is added a strong acid such as trifluoroacetic acid. The resultant solution is maintained at ambient temperature overnight and concentrated under reduced pressure. The residue is isolated to give a compound of Formula 705 which is used in the subsequent step without purification.

Preparation of Formula 707

Referring to Reaction Scheme 4, Step 3, to a solution of a compound of Formula 705 in a polar, aprotic solvent such as dichloromethane is added an excess, such as about two equivalents of an amine base such as triethylamine, followed by about an equivalent or slight excess of an acid chloride. The resultant solution is stirred at ambient temperature for about 3 hours. Completion is monitored, e.g., by TLC. The corresponding compound of Formula 707 is isolated and purified.

Preparation of Formula 709

Referring to Reaction Scheme 7, Step 4, a solution of a compound of Formula 707 in an excess of phosphorus oxychloride is heated at reflux. After 8 hours, the reaction mixture is allowed to cool to ambient temperature and concentrated under reduced pressure. The corresponding compound of Formula 709 is isolated and purified. 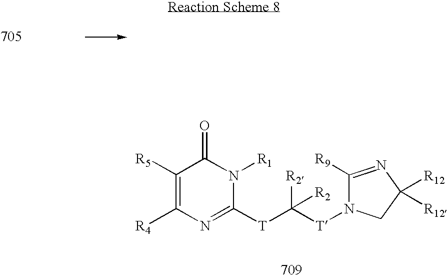
Preparation of Formula 709

As an alternative to Steps 3 and 4 of Reaction Scheme 7, acylation of primary amines of Formula 705, followed by acetic acid mediated cyclization, can proceed without isolation of the intermediate amides to provide the target compound of Formula 709. This route is shown in Reaction Scheme 8.

More specifically, to a solution of a compound of Formula 705 in a polar, aprotic solvent such as dichloromethane is added an excess, such as about two equivalents of an amine base, such as triethylamine, followed by about an equivalent of an acid chloride. The resultant solution is stirred at ambient temperature for 2 hours, then evaporated under reduced pressure. The resultant solid is treated with glacial acetic acid, then the resultant suspension is heated at reflux for about 48 hours. The reaction is cooled to ambient temperature then evaporated under reduced pressure. The corresponding compound of Formula 709 is isolated and purified. 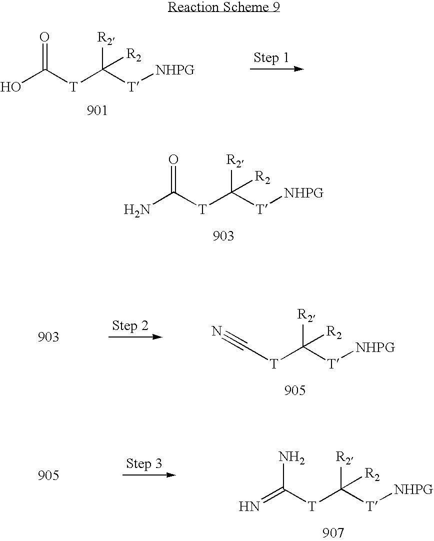
Preparation of Compounds of Formula 903

Referring to Reaction Scheme 9, Step 1, to a 0° C. solution of a compound of Formula 901 and an excess (such as about 1.4 equivalents) of N-methyl morpholine in an anhydrous, nonpolar, aprotic solvent such tetrahydrofuran is added an excess (such as about 1.3 equivalents) of iso-butyl chloroformate. The resulting mixture is stirred at room temperature for about 4 hours. The flask is then equipped with a dry-ice reflux condenser and purged continuously with gaseous ammonia for about 2 hours. The resulting reaction mixture is then stirred at room temperature overnight. The product, a compound of Formula 903, is isolated and used without further purification.

Preparation of Compounds of Formula 905

Referring to Reaction Scheme 9, Step 2, to a room temperature solution of a compound of Formula 903 in a nonpolar, aprotic solvent such as dioxane is added an excess (such as about 2.5 equivalents) of pyridine and an excess (such as about 2 equivalents) of trifluoroacetic anhydride, successively. The resulting solution is stirred for about 4 hours until no sarting material is present. The product, a compound of Formula 905, is isolated and purified.

Preparation of Compounds of Formula 907

Referring to Reaction Scheme 9, Step 3, to a room temperature solution of a compound of Formula 905 and N-acetylcysteine in a polar, protic solvent such as ethylene glycol is added solid ammonium acetate. The resulting solution is heated to about 100° C. for about 48 hours. Most of the ethylene glycol is distilled in vacuo. The product, a compound of Formula 907, is isolated and used without further purification. 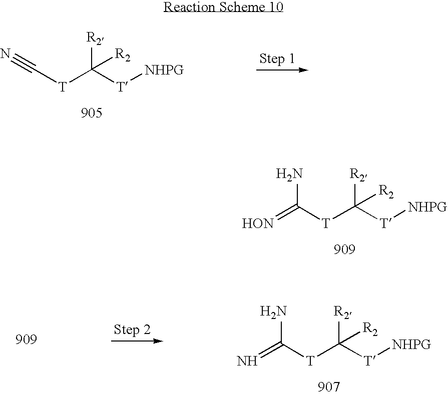
Preparation of Compounds of Formula 909

Referring to Reaction Scheme 10, Step 1, a solution of sodium methoxide in methanol (such as about 2 equivalents of a 0.5 M solution) is then added to a compound of Formula 905. To the resulting reaction mixture is added an excess (such as about 2 equivalents) of hydroxylamine hydrochloride. The reaction mixture is then heated to about 50° C. overnight. The product, a compound of Formula 909, is isolated and used without further purification.

Preparation of Compounds of Formula 907

To a room temperature solution of a compound of Formula 909 in acetic acid is added an excess (such as about 1.5 equivalents) of acetic anhydride and Pd/C. The reaction mixture is stirred under a hydrogen atmosphere for about 24 hours and then filtered through Celite. The product, a compound of Formula 907, is isolated and used without further purification. 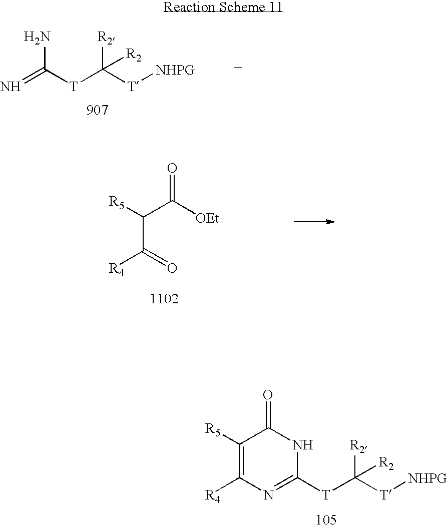
Preparation of Compounds of Formula 1103

Referring to Reaction Scheme 11, to a solution of a compound of Formula 1102 and a compound of Formula 907 is added a solution of sodium methoxide in methanol (such as about 2.4 equivalents of a 0.5 M solution). The resulting solution is heated to about 60° C. for about 30 minutes. The product, a compound of Formula 1103, is isolated and purified. 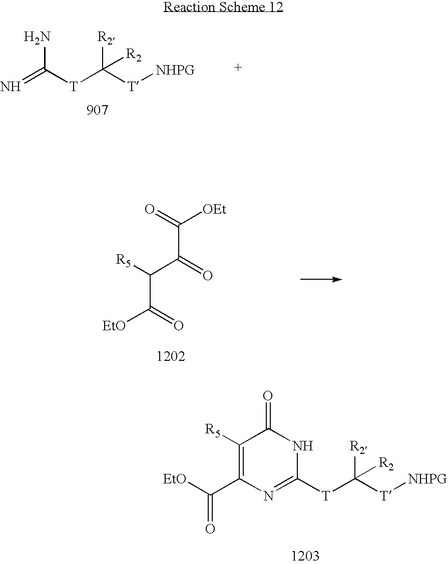
Preparation of Compounds of Formula 1203

Referring to Reaction Scheme 12, to a room temperature solution of a compound of Formula 907 and about an equivalent of diisopropylethylamine in anhydrous ethanol is added about an equivalent of a compound of Formula 1202. The resulting mixture is heated to about 70° C. for about 16 hours. The product, a compound of Formula 1203, is isolated and purified. 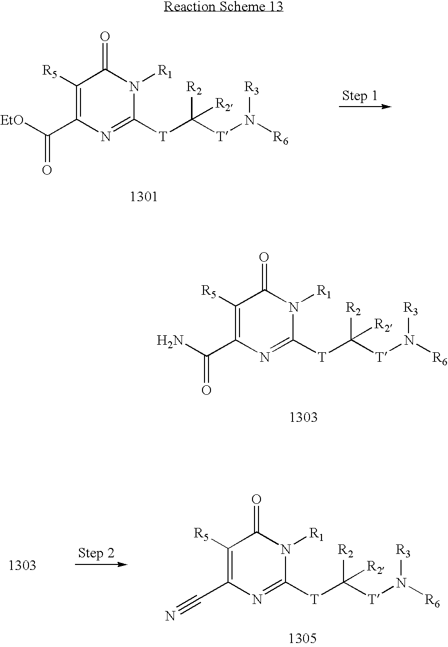
Preparation of Compounds of Formula 1303

Referring to Reaction Scheme 13, Step 1, to a room temperature solution of a compound of Formula 1301 in a polar, protic solvent such as ethanol is added concentrated aqueous ammonia. The resulting mixture is stirred at room temperature for about 24 hours. The product, a compound of Formula 1303, is isolated and purified.

Preparation of Compounds of Formula 1305

Referring to Reaction Scheme 13, Step 2, to a room temperature solution of a compound of Formula 1303 in pyridine is added an excess of thionyl chloride. The product, a compound of Formula 1305, is isolated and purified. 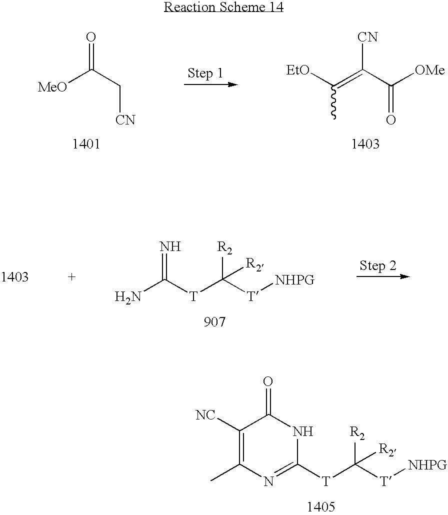
Preparation of Compounds of Formula 1403

Referring to Reaction Scheme 14, Step 1, a solution of methyl cyanoacetate of Formula 1401 and an excess (such as about 1.1 eqivalents) of triethylorthoacetate in acetic anhydride is heated to reflux for about 3 hours. The product, a compound of Formula 1403, is isolated and used without further purification.

Preparation of Compounds of Formula 1405

Referring to Reaction Scheme 14, Step 2, to a solution of a compound of Formula 1403 and an excess (such as about 3 equivalents) of a compound of Formula 907 in a polar, protic solvent such as methanol is added a solution of sodium methoxide in methanol (such as a 0.5 M solution). The resulting solution is stirred at about 60° C. under an atmosphere of nitrogen for about 30 minutes. The product, a compound of Formula 1405, is isolated and purified.

Alternative Preparation of Compounds of Formula 1405

Referring to Reaction Scheme 14, Step 2, alternatively, to a stirred solution of a compound of Formula 907 in a polar, protic solvent such as ethanol is added about an equivalent of a compound of Formula 1403. The reaction is refluxed for about 18 h and cooled to RT. The product, a compound of Formula 1405, is isolated and purified. 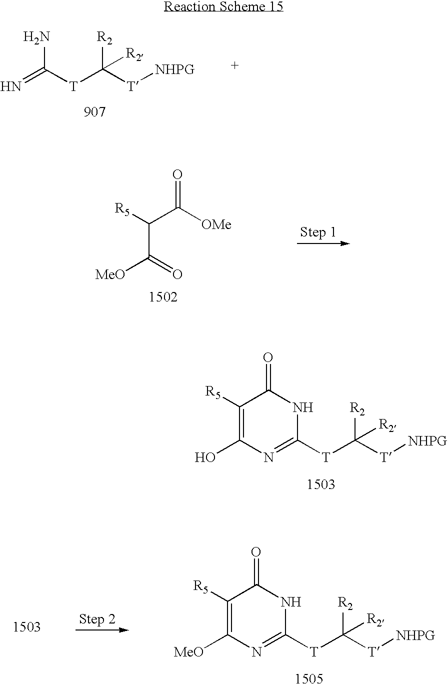
Preparation of Compounds of Formula 1503

Referring to Reaction Scheme 15, Step 1, to a room temperature solution of a compound of Formula 1502, such as an optionally substituted dialkyl malonate and an excess (such as about 1.5 equivalents) of a compound of Formula 907 in methanol is added a solution of an excess of sodium methoxide in methanol (such as as a 0.5 M solution in methanol). The resulting solution is heated to about 60° C. for about 4 hours. The product, a compound of Formula 1503, is isolated and used without further purification.

Preparation of Compounds of Formula 1505

Referring to Reaction Scheme 15, Step 2, to a solution of a compound of Formula 1503 in a nonopolar, aprotic solvent such as DMF is added sodium bicarbonate and dimethyl sulfate. The resulting solution is stirred at about 0° C. for about 4 hours. The product, a compound of Formula 1505, is isolated and purified. 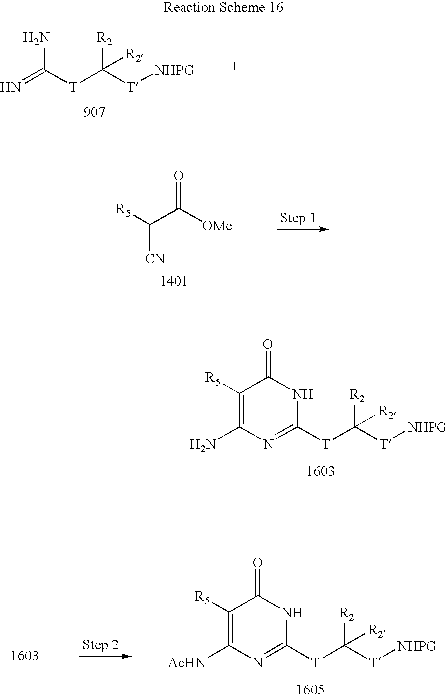
Preparation of Compounds of Formula 1603

Referring to Reaction Scheme 16, Step 1, to a room temperature solution of methyl cyanoacetate (i.e., compound of Formula 1401) and an excess (such as about 1.5 equivalents) of a compound of Formula 907 in methanol is added a solution of sodium methoxide in methanol (such as about 1.8 equivalents of a 0.5 M solution in methanol). The resulting solution is heated to about 60° C. for about 4 hours. The product, a compound of Formula 1603, is isolated and used without further purification.

Preparation of Compounds of Formula 1605

To an about 0° C. solution of a compound of Formula 1603 in a nonpolar, aprotic solvent such as tetrahydrofuran are successively added diisopropylethylamine and an excess (such as about 2 equivalents) of an acid chloride (e.g., acetyl chloride). The resulting solution is stirred at about 0° C. for about 6 hours. The product, a compound of Formula 1605, is isolated and purified. 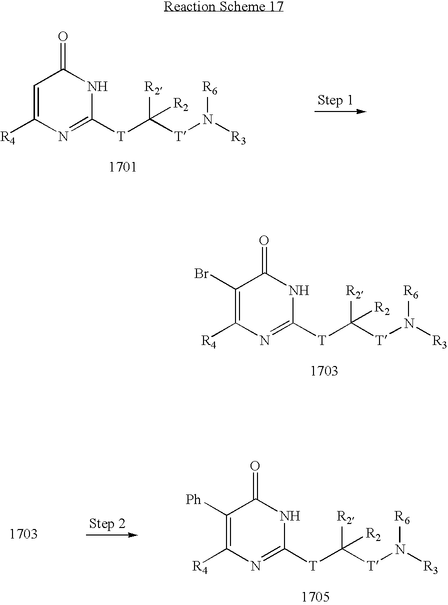
Preparation of Compounds of Formula 1703

Referring to Reaction Scheme 17, Step 1, to a room temperature solution of a compound of Formula 1701 in carbon tetrachloride is added about an equivalent of N-bromosuccinimide. The resulting mixture is heated to about 85° C. for about 1 hour. The product, a compound of Formula 1703, is isolated and purified.

Preparation of Compounds of Formula 1705

Referring to Reaction Scheme 17, Step 2, a compound of Formula 1703, about 0.2 equivalent of 2-(dicyclohexylphosphino)biphenyl, about 0.1 equivalent of palladium acetate, an excess (such as about 1.5 equivalents) of phenylboronic acid, and an excess (such as about 3 equivalents) of potassium fluoride are placed in a resealable Schlenk tube. The tube is evacuated and back-filled with nitrogen several times. Toluene is then added by syringe, and the resulting mixture is heated to about 80° C. for about 72 h. The product, a compound of Formula 1705, is isolated and purified.

Alternative Preparation of Compounds of Formula 1705

Referring again to Reaction Scheme 17, Step 2, a 10-mL Smith microwave reaction vial is charged with a compound of Formula 1703, about an equivalent of 3-chloroboronic acid, Na₂CO₃, and PdCl₂(PPh₃)₂ followed by MeCN—H₂O (1:1). The mixture is purged with argon gas, sealed, and subjected to the microwave reactor for about 5 min at about 150 □C. The product, a compound of Formula 1705, is isolated and purified. 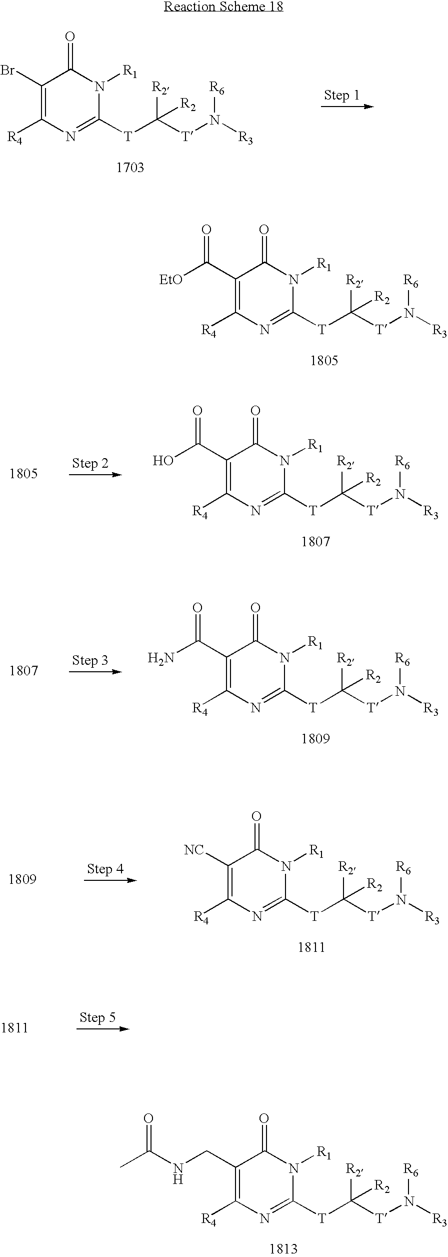
Preparation of Compounds of Formula 1805

Referring to Reaction Scheme 18, Step 1, to a solution of a compound of Formula 1703 in anhydrous ethanol in a thick-walled glass tube is added about 0.25 equivalent of 1,3-bis(diphenylphosphino)propane, an excess of triethylamine and about 0.2 equivalent of palladium acetate. The tube is evacuated and back-filled with carbon monoxide three times and then pressurized with carbon monoxide (at about 30 psi). The mixture is heated to about 70° C. for about 48 hours. The product, a compound of Formula 1805, is isolated and purified.

Preparation of Compounds of Formula 1807

Referring to Reaction Scheme 18, Step 2, to a room temperature solution of a compound of Formula 1805 in tetrahydrofuran and methanol is added aqueous potassium hydroxide. The resulting mixture is heated to about 70° C. for about 4 hours. The product, a compound of Formula 1807, is isolated and used without further purification.

Preparation of Compounds of Formula 1809

Referring to Reaction Scheme 18, Step 3, to a room temperature solution of a compound of Formula 1807 in anhydrous tetrahydrofuran are successively added an excess (such as about 3 equivalents) of diisopropylethylamine and an excess (such as about 1.2 equivalents) of isobutyl chloroformate. The resulting mixture is stirred for about 3 hours at room temperature under an atmosphere of nitrogen. The reaction is then cooled to about 0° C. and purged with gaseous ammonia for about 45 minutes. The mixture is then allowed to warm to room temperature for an addition 45 minutes. The product, a compound of Formula 1809, is isolated and purified.

Preparation of Compounds of Formula 1811

Referring to Reaction Scheme 18, Step 4, to a room temperature solution of a compound of Formula 1809 in pyridine is added thionyl chloride. The reaction mixture is stirred at room temperature for about 16 hours. The product, a compound of Formula 1811, is isolated and purified.

Preparation of Compounds of Formula 1813

Referring to Reaction Scheme 18, Step 5, to a stirred solution of a compound of Formula 1811 in acetic acid is carefully added 10% PDd/C. The reaction is hydrogenated under a balloon of hydrogen for about 18 hours at RT and the crude amine is isolated. To the crude amine in a nonpolar, aprotic solvent such as CH₂Cl₂ is added with stirring a base such as triethylamine and acetic anhydride. After stirring at RT for about 2 hours, the product, a compound of Formula 1813, is isolated and purified. 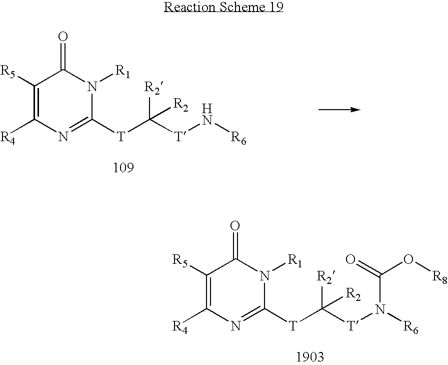
Preparation of Compounds of Formula 1903

Referring to Reaction Scheme 19, a compound of Formula 109 is reacted with a slight excess of a compound of the formula R₈O(CO)Cl in the presence of a base such as triethylamine in a nonpolar, aprotic solvent such as dichloromethane. The product, a compound of Formula 1903 is isolated and purified. 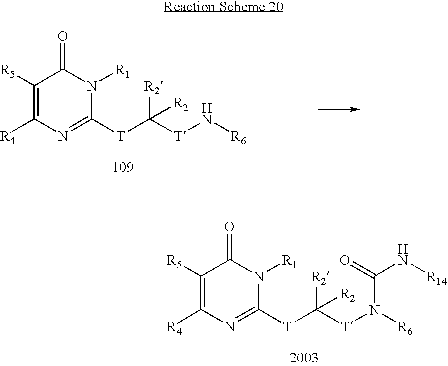
Preparation of Compounds of Formula 2003

Referring to Reaction Scheme 20, a compound of Formula 109 is treated with a slight excess of an isocyanate R₁₄—N═C═O in the presence of a base, such as triethylamine, in a nonpolar, aprotic solvent, such as dichloromethane. The product, a compound of Formula 2003, is isolated and purified. 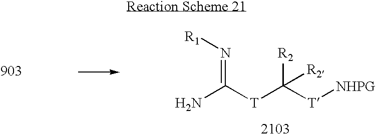
Preparation of Compounds of Formula 2103

Referring to Reaction Scheme 21, to a stirred solution of a compound of Formula 903 in a nonpolar, aprotic solvent such as CH₂Cl₂ is added an excess (such as about 1.05 equivalents) of triethyloxonium hexafluorophosphate. The reaction is stirred for about 48 h at RT, poured into a separatory funnel, washed, dried, filtered and concentrated under vacuum. To the remaining oil is added a compound of formula R₁NH₂ and a polar, protic solvent such as ethanol. The reaction is stirred at about 60° C. for about 24 h. The product, a compound of Formula 2103, is isolated and used without purification. 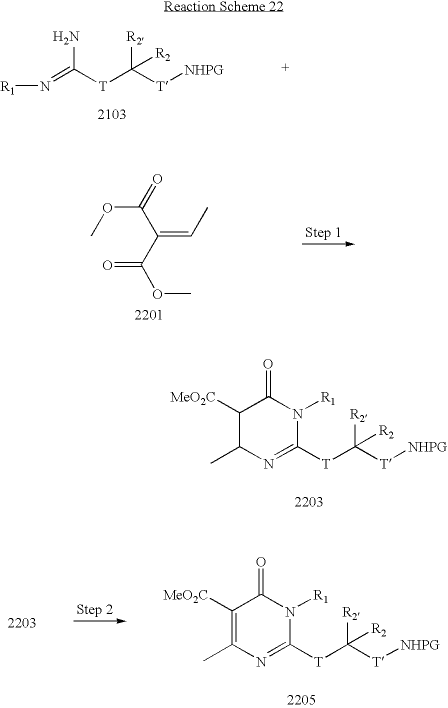
Preparation of Compounds of Formula 2203

Referring to Reaction Scheme 22, Step 1, to a solution of a compound of Formula 2103 in a polar, protic solvent such as methanol is added about an equivalent of a compound of Formula 2201 (i.e., dimethyl ethylidenemalonate). The reaction is slowly heated to about 110° C. allowing the solvent to distill off. The reaction is stirred for about 5 h at about 110° C. then allowed to cool to RT. The product, a compound of Formula 2203, is isolated and purified.

Preparation of Compounds of Formula 2205

Referring to Reaction Scheme 22, Step 2, to a stirred solution of a compound of Formula 2203 in a nonpolar, aprotic solvent such as CCl₄ is added K₂CO₃, N-bromosuccinimde, and benzoyl peroxide. The reaction is refluxed for about 0.5 h and cooled to RT. The product, a compound of Formula 2205, is isolated and purified. 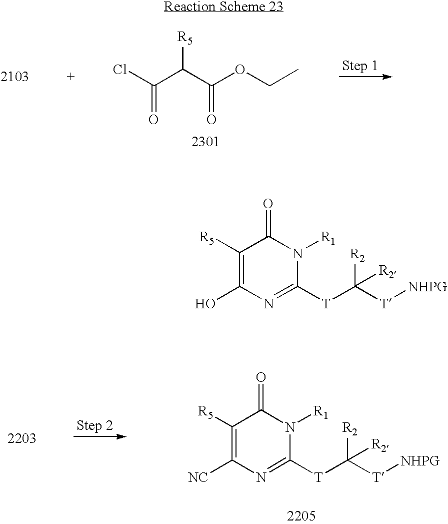
Preparation of Compounds of Formula 2303

Referring to Reaction Scheme 23, Step 1, to a stirred solution of a compound of Formula 2103 in a nonpolar, aprotic solvent such as CH₂Cl₂ with cooling at about 0° C. is added a base such as Et₃N followed by an excess (such as about 1.1 equivalent) of a compound of Formula 2301 (such as wherein R₅ is methyl) dropwise over about 15 minutes. The reaction is allowed to warm to RT and stirred for about 4 h. The product, a compound of Formula 2303, is isolated and purified.

Preparation of Compounds of Formula 2305

Referring to Reaction Scheme 23, Step 2, to a stirred solution of a compound of Formula 2303 is added portionwise a 60% dispersion of NaH in mineral oil. After stirring for about 15 minutes at RT, an excess (such as about 1.1 equivalents) of N-phenyltrifluoromethanesulfonimide is added. The reaction is stirred at RT for about 18 h. The product, the corresponding triflate, is isolated and purified. To the crude triflate with stirring in a nonpolar, aprotic solvent such as DMF is added Zn(CN)₂ and (PPh₃)₄Pd. The reaction is heated under an inert atmosphere at about 90° C. for about 4 h and cooled to RT. The product, a compound of Formula 2305, is isolated and purified.

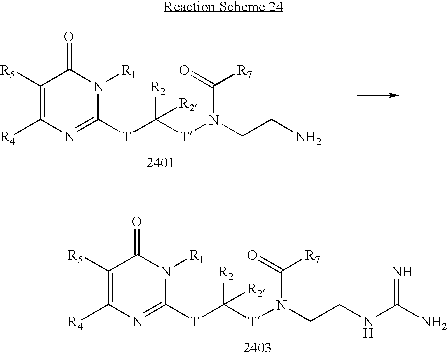

To a solution of a compound of Formula 2401 in a nonpolar, aprotic solvent such as DMF is added 1H-pyrazole-1-carboxamidine hydrochloride and diisopropylethyl amine. The reaction is stirred at RT for about 16 h. The product, a compound of 2403, is isolated and purified. 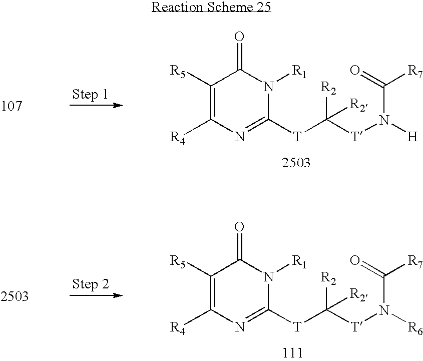
Preparation of Compounds of Formula 2503

Referring to Reaction Scheme 25, Step 1, to a compound of Formula 107 and a base such as triethylamine in a nonpolar, aprotic solvent such as CH₂Cl₂ is added a compound of Formula R₇—(CO)Cl. The reaction is stirred at RT for about 48 h. The product, a compound of Formula 2503, is isolated and purified.

Preparation of Compounds of Formula 111

Referring to Reaction Scheme 25, Step 2, to a compound of Formula 2503 in a nonpolar, aprotic solvent such as DMF is added a base, such as sodium hydride. The reaction is stirred for about 15 at RT then a compound of Formula R₆—X wherein X is a leaving group (such as a halide) is added. The reaction is stirred at RT for about 24 h. The product, a compound of Formula 111, is isolated and purified. 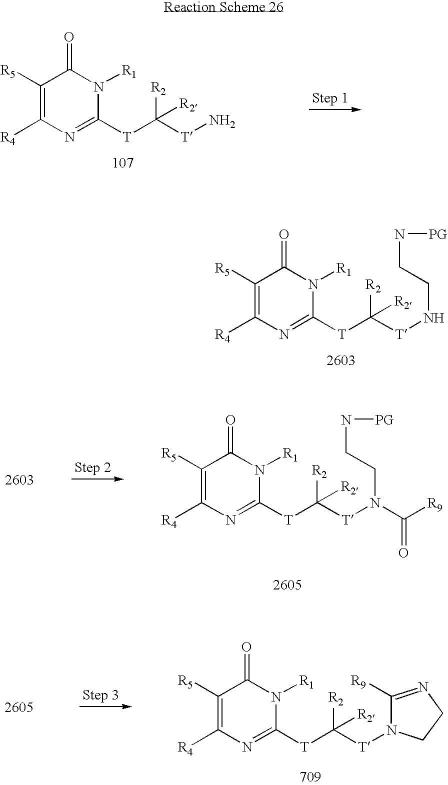
Preparation of Compounds of Formula 2603

Referring to Reaction Scheme 26, Step 1, a compound of Formula 107 and an excess of a compound of Formula PG-N—CH₂CHO (such as 2H-isoindole-2-acetaldehyde) are dissolved in a nonpolar, aprotic solvent such as dichloroethane. Glacial acetic acid is added followed by sodium triacetoxy borohydride. The reaction is stirred at room temperature under nitrogen for about 3.5 h. The product, a compound of Formula 2603, is isolated and purified.

Preparation of Compounds of Formula 2605

Referring to Reaction Scheme 26, Step 2, a slight excess (such as about 1.1 equivalents) of a compound of the Formula R₉—(CO)—Cl is dissolved in a nonpolar, aprotic solvent such as toluene and is treated with a base followed by a compound of Formula 2603. The reaction is stirred at about 110° C. for about 3 h. The reaction is cooled to room temperature. The product, a compound of Formula 2605, is isolated and purified.

Preparation of Compounds of Formula 709

Referring to Reaction Scheme 26, Step 3, to a solution of a compound of Formula 2605 in a polar, aprotic solvent such as dichloromethane is added a strong acid such as trifluoroacetic acid. The resultant solution is maintained at ambient temperature overnight and concentrated under reduced pressure. The residue is isolated to give the corresponding free amine which is used in the subsequent step without purification.

A solution of the free amine prepared above in an excess of phosphorus oxychloride is heated at reflux. After 8 hours, the reaction mixture is allowed to cool to ambient temperature and concentrated under reduced pressure. The corresponding compound of Formula 709 is isolated and purified. 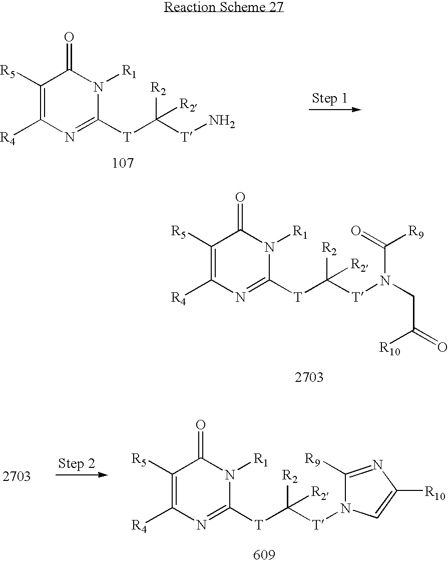
Preparation of Compounds of Formula 2703

Referring to Reaction Scheme 27, Step 1, to a compound of Formula 107 in a nonpolar, aprotic solvent such as DMF is added a compound of Formula X—CH₂—(CO)—R₁₀ (wherein X is a leaving group, such as a halide) and a base such as N,N-diisopropylethylamine. The reaction is stirred for about 16 h at room temperature. The product is isolated and added to a nonpolar, aprotic solvent such as triethylamine and a compound of the formula R₉—(CO)—Cl. The reaction is stirred for about 16 h at room temperature. The product, a compound of Formula 2703, is isolated and purified.

Preparation of Compounds of Formula 609

Referring to Reaction Scheme 27, Step 2, to a compound of Formula 2703 in glacial acetic acid is added ammonium acetate and the reaction is heated at reflux for about 16 h. The product, a compound of Formula 609, is isolated and purified. 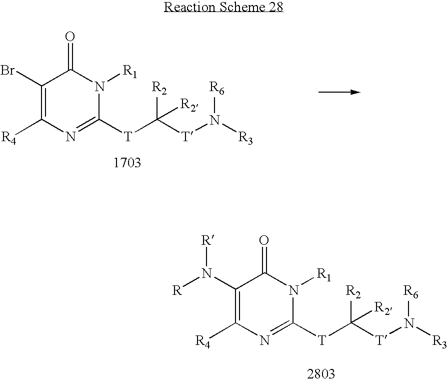
Preparation of Compounds of Formula 2803

Referring to Reaction Scheme 28, to a compound of Formula 1703 in a nonpolar, aprotic solvent such as toluene is added an amine of the formula H—NRR′, a base such as NaO-tBu, Pd₂DBA, and (S)-BINAP. The reaction is stirred at about 90° C. for about 72 h. The product, a compound of Formula 2803, is isolated and purified.

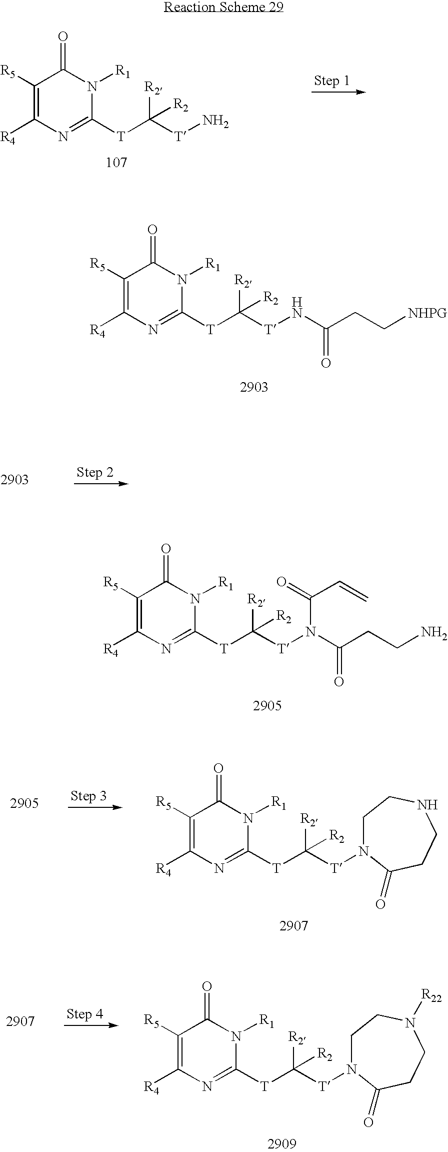

Referring to Reaction Scheme 29, acylation of 107 with protected aminopropionic acid gives the corresponding amide. Acylation with acryloyl chloride followed by deprotection of the primary amide and base mediated cyclisation gave the desired diazepanones. If desired, further functionalization of the basic amine could be accomplished under conditions well known to those skilled in the art.

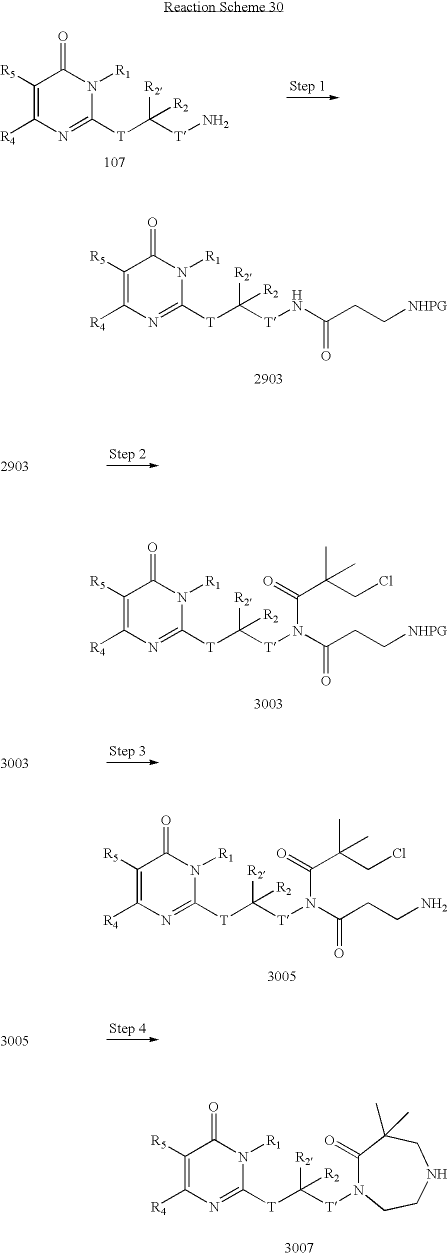

Referring to Reaction Scheme 30, reductive amination of the primary amino group in compounds of Formula 107 with (2-oxo-ethyl)-carbamic acid tert-butyl ester gave the corresponding secondary amide. Acylation with chloropivaloyl chloride followed by deprotection of the primary amide and base mediated cyclisation gave the desired diazepanones. If desired, further functionalization of the basic amine could be accomplished under conditions well known to those skilled in the art.

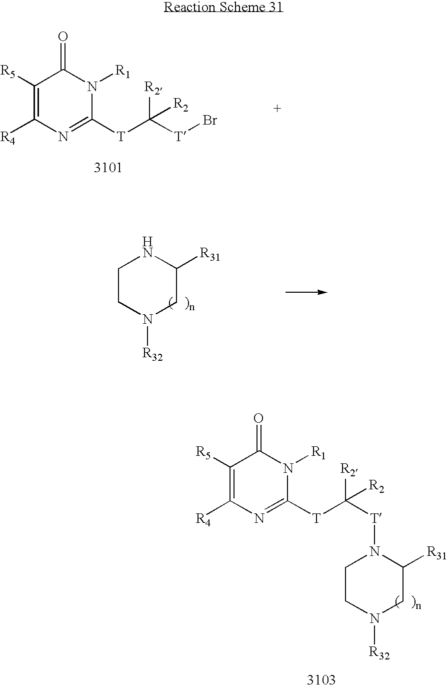

Referring to Reaction Scheme 31, a compound of Formula 3101, one-half molar equivalent of an optionally substituted piperazine or diazepam (as shown above, where R₃₂ is as described herein) and an excess of potassium carbonate are combined in an organic solvent (e.g., acetonitrile). The reaction takes place under a nitrogen atmosphere at elevated temperature (e.g., 100° C.) over a period of 8 hours, followed at a somewhat lower temperature (e.g., 60° C.) for a period of 5 days. The product, a compound of Formula 3103, is isolated and purified.

Optionally, in the event that R₃₂ is an amine protecting group, such as Boc, it may be removed by for example treatment with a 95/5 mixture of TFA/water followed by stirring at room temperature for 1 hour. The product, a compound of Formula 3103 wherein R₃₂ is hydrogen, can be isolated and purified. If desired, further functionalization of the basic amine could be accomplished under conditions well known to those skilled in the art.

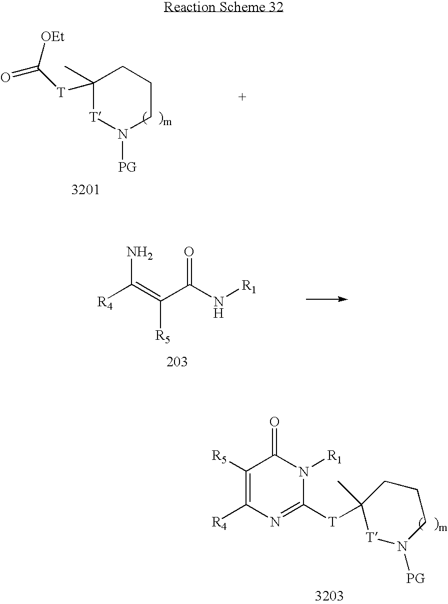

The synthesis of compounds of Formula I wherein R₆ taken together with R₂ form an optionally substituted 5- to 12-membered nitrogen containing heterocycle can be accomplished according to the general procedure shown above and as described in Reaction Scheme 2.

A compound of Formula I is optionally contacted with a pharmaceutically acceptable acid or base to form the corresponding acid or base addition salt.

A pharmaceutically acceptable acid addition salt of a compound of Formula I is optionally contacted with a base to form the corresponding free base of Formula I.

A pharmaceutically acceptable base addition salt of a compound of Formula I is optionally contacted with an acid to form the corresponding free acid of Formula I.

T and T′

When considering the compounds of Formula I, T is optionally substituted alkylene or is absent; and T′ is optionally substituted alkylene or is absent. In some embodiments, one of T and T′ is absent and the other is optionally substituted alkylene (such as optionally substituted methylene). In some embodiments, both are absent. In some embodiments, both are optionally substituted alkylene.

R₁

When considering the compounds of Formula I, in some embodiments, R₁ is selected from hydrogen, optionally substituted C₁-C₈ alkyl-, optionally substituted aryl-, optionally substituted heteroaryl-, optionally substituted aryl-C₁-C₄-alkyl-, and optionally substituted heteroaryl-C₁-C₄-alkyl-. In some embodiments, R₁ is selected from hydrogen, optionally substituted C₁-C₄ alkyl-, optionally substituted phenyl-C₁-C₄-alkyl-, optionally substituted heteroaryl-C₁-C₄ alkyl, optionally substituted naphthalenylmethyl-, optionally substituted phenyl-, and naphthyl-. In some embodiments, R₁ is optionally substituted phenyl-C₁-C₄-alkyl-, optionally substituted heteroaryl-C₁-C₄-alkyl-, optionally substituted naphthalenylmethyl-, optionally substituted phenyl, or naphthyl.

In some embodiments, R₁ is naphthyl-, phenyl-, bromophenyl-, chlorophenyl-, methoxyphenyl-, ethoxyphenyl-, tolyl-, dimethylphenyl-, chorofluorophenyl-, methylchlorophenyl-, ethylphenyl-, phenethyl-, benzyl-, halobenzyl-(such as chlorobenzyl or bromobenzyl), methylbenzyl-, methoxybenzyl-, cyanobenzyl-, hydroxybenzyl-, dichlorobenzyl-, dimethoxybenzyl-, or naphthalenylmethyl-.

In some embodiments, R₁ is optionally substituted phenyl-C₁-C₄ alkyl or optionally substituted heteroaryl-C₁-C₄ alkyl. In some embodiments, R₁ is benzyl, halobenzyl, methylbenzyl, hydroxybenzyl, cyanobenzyl, methoxybenzyl, or naphthalenylmethyl-. In some embodiments, R₁ is benzyl-.

R₂ and R_{2′}

When considering the compounds of Formula I and as will be appreciated by those skilled in the art, the compounds described herein possess a potentially chiral center at the carbon to which R₂ and R_{2′} are attached. The R₂ and R_{2′} groups may be the same or different; if different, the compound is chiral (i.e., has a stereogenic center). When R₂ and R_{2′} are different, in some embodiments R_{2′} is hydrogen and R₂ is other than hydrogen. The invention contemplates the use of pure enantiomers and mixtures of enantiomers, including racemic mixtures, although the use of a substantially optically pure enantiomer will generally be preferred. The term “substantially pure” means having at least about 95% chemical purity with no single impurity greater than about 1%. The term “substantially optically pure” or “enantiomerically pure” means having at least about 97.5% enantiomeric excess. In some embodiments, the stereogenic center to which R₂ and R_{2′} are attached is of the R configuration.

When considering the compounds of Formula I, R₂ and R_{2′} are independently chosen from hydrogen, optionally substituted alkyl-, optionally substituted alkoxy, optionally substituted aryl-, optionally substituted aralkyl-, optionally substituted heteroaryl-, and optionally substituted heteroaralkyl-; or R₂ and R₂ taken together form an optionally substituted 3- to 7-membered ring.

In some embodiments, R₂ is optionally substituted C₁-C₄ alkyl-, and R₂ is hydrogen or optionally substituted C₁-C₄ alkyl-. More suitably, R_{2′} is hydrogen and R₂ is optionally substituted C₁-C₄ alkyl-. In some embodiments, R₂ is chosen from methyl-, ethyl-, propyl (such as c-propyl or i-propyl), butyl (such as t-butyl), methylthioethyl-, methylthiomethyl-, aminobutyl-, (CBZ)aminobutyl-, cyclohexylmethyl-, benzyloxymethyl-, methylsulfanylethyl-, methylsulfanylmethyl-, and hydroxymethyl-, and R_{2′} is hydrogen. In some embodiments, R_{2′} is hydrogen and R₂ is ethyl or propyl (such as c-propyl or i-propyl). In some embodiments, R₂ is i-propyl. In some embodiments, the stereogenic center to which R₂ and R_{2′} is attached is of the R configuration.

In some embodiments, if either R₂ or R_{2′} is hydrogen, then the other is not hydrogen. In some embodiments, both R₂ and R_{2′} are hydrogen.

R₂ Taken Together with R₆

When considering the compounds of Formula I, in some embodiments, R₂ and R₆ taken together form a 5- to 12-membered ring which optionally incorporates one or two additional heteroatoms, selected from N, O, and S in the heterocycle ring and may optionally be substituted with one or more of the following groups: alkyl, aryl, aralkyl, heteroaryl, substituted alkyl, subtituted aryl, substituted aralkyl, substituted heteroaryl, hydroxy, alkoxy, cyano, optionally substituted amino, and oxo.

In some embodiments, R₂ and R₆ taken together form an optionally subtituted ring of the formula: 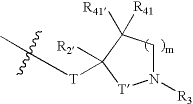
wherein R₄₁, and R_{41′}, are independently chosen from hydrogen, alkyl, aryl, aralkyl, heteroaryl, substituted alkyl, substituted aryl, substituted aralkyl, and substituted heteroaryl; m is 0, 1, 2, or 3; and T, T′, R₃, and R_{2′}, are as defined herein. In some embodiments, R₄₁ is hydrogen. In some embodiments, both R₄₁ and R_{41′} are hydrogen. In some embodiments, R₃ is optionally substituted aralkyl (such as benzyl) or optionally substituted acyl (i.e., R₃ is —(CO)R₇ where R₇ is as defined herein, such as where R₇ is optionally subsituted phenyl). See, e.g., WO 2004/034972, which is incorporated herein by reference for all purposes.

In some embodiments, R₂ and R₆ taken together form an optionally substituted ring of the formula: 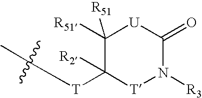
wherein R₃, R_{2′}, T, and T′ are as defined herein; R₅₁ and R_{51′} are independently chosen from hydrogen, alkyl, aryl, aralkyl, heteroaryl, substituted alkyl, substituted aryl, substituted aralkyl and substituted heteroaryl; U is a covalent bond, CR′R″ or NR′″; R′ and R″ are independently chosen from hydrogen, hydroxy, amino, optionally substituted aryl, optionally substituted alkylamino, optionally substituted alkyl and optionally substituted alkoxy; and R′″ is chosen from hydrogen, optionally substituted alkyl, optionally substituted aryl, optionally substituted aralkyl, optionally substituted heteroaryl, and optionally substituted heteroaralkyl.

In some embodiments, R₅₁ is hydrogen or optionally substituted lower alkyl; in some embodiments, R₅₁ is hydrogen. In some embodiments, R_{51′} is hydrogen or optionally substituted lower alkyl; in some embodiments, R_{51′} is hydrogen.

In some embodiments, R₃ is optionally substituted aryl or optionally substituted aralkyl; in some embodiments, R₃ is optionally substituted phenyl, benzyl or methyl-benzyl (such as benzyl or methyl-benzyl).

In some embodiments, U is CR′R″ where R′ and/or R″ are hydrogen. In some embodiments, U is NR′″ where R′″ is hydrogen or optionally substituted alkyl. In some embodiments, R′″ is hydrogen or optionally substituted amino-lower alkyl. See, e.g., US 2004-0142949, which is incorporated herein by reference for all purposes.

R₄ and R₅

When considering the compounds of Formula I, in some embodiments, R₄ is chosen from hydrogen, optionally substituted alkyl, optionally substituted alkoxy, acyl, halogen, hydroxy, nitro, cyano, carboxy, sulfonyl, sulfanyl, aminocarbonyl, optionally substituted amino, optionally substituted aryl, optionally substituted aralkyl, optionally substituted heteroaralkyl and optionally substituted heteroaryl. In some embodiments, R₄ is hydrogen, acyl, alkoxy, cyano, carboxy, optionally substituted amino, aminocarbonyl, lower-alkyl, lower-alkyl substituted with one or more of the following substituents: halo, lower-alkoxy, or hydroxy, phenyl, or phenyl substituted with one or more of the following substituents: halo, lower-alkoxy, or hydroxy. In some embodiments, R₄ is hydrogen, cyano, methyl, or methyl substituted with one or more of the following substituents: halo, lower-alkoxy, or hydroxy (such as halo, for example, trifluoromethyl).

When considering the compounds of Formula I, in some embodiments, R₅ is chosen from hydrogen, optionally substituted alkyl, optionally substituted alkoxy, acyl, halogen, hydroxy, nitro, cyano, sulfonyl, sulfanyl, aminocarbonyl, optionally substituted amino, optionally substituted aryl, optionally substituted aralkyl, optionally substituted heteroaralkyl and optionally substituted heteroaryl.

In some embodiments, R₅ is hydrogen, acyl, carboxy, aminocarbonyl, optionally substituted amino, cyano, lower-alkyl (such as methyl or ethyl), halo (such as bromo, chloro or fluoro), benzyl, piperonyl, naphthyl, furyl, thienyl, indolyl, morpholinyl, phenyl, benzodioxolyl, or phenyl substituted with one or more of the following substituents: optionally substituted amino, aminocarbonyl, cyano, halo, optionally substituted lower-alkyl-(including trifluoromethyl and hydroxy alkyl such as hydroxymethyl), optionally substituted lower-alkoxy, optionally substituted lower-alkyl sulfanyl (including methylsulfanyl), hydroxy, or thio.

In some embodiments, R₅ is hydrogen; methyl; ethyl; bromo; carboxy; cyano; phenyl; halophenyl; lower-alkylphenyl; trifluoromethylphenyl; lower-alkoxyphenyl; di(lower-alkoxy)phenyl; polyhalophenyl; halo lower-alkylphenyl (e.g., halomethylphenyl); furyl; thienyl; lower-alkylsulfanylphenyl; thiophenyl; aminophenyl; aminocarbonylphenyl; cyanophenyl; di(lower-alkyl)aminophenyl; di(lower-alkyl)phenyl; acetylaminophenyl; amino substituted lower-alkylphenyl; hydroxy substituted lower-alkylphenyl (e.g., methylhydroxyphenyl); piperonyl; naphthyl; carbamoyl; lower-alkyl carbamoyl (e.g., methyl, ethyl, or propyl carbamoyl); benzylcarbamoyl; phenylcarbamoyl; methoxymethyl carbamoyl; methoxyethyl carbamoyl; hydroxymethyl carbamoyl; hydroxyethyl carbamoyl; indolyl; morpholinyl; and morpholinocarbonyl.

In some embodiments, R₅ is hydrogen, methyl, or cyano.

R₃ Taken Together with R₆

When considering the compounds of Formula I, in some embodiments, R₃ taken together with R₆, and the nitrogen to which they are bound, form an optionally substituted 5- to 12-membered nitrogen-containing heterocycle, which optionally incorporates from one to two additional heteroatoms, selected from N, O, and S, in the heterocycle ring and may optionally be substituted with one or more of the following groups: alkyl, aryl, aralkyl, heteroaryl, substituted alkyl, substituted aryl, substituted aralkyl, substituted heteroaryl, hydroxy, alkoxy, cyano, optionally substituted amino, and oxo.

In some embodiments, when T and T′ are not both absent, R₃ taken together with R₆ and the nitrogen to which they are bound, form an optionally substituted imidazolyl ring of the formula: 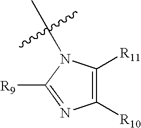
wherein

R₉ is chosen from hydrogen, optionally substituted C₁-C₈ alkyl-, optionally substituted aryl-, optionally substituted aryl-C₁-C₄-alkyl-, optionally substituted heteroaryl-C₁-C₄-alkyl-, optionally substituted aryl-C₁-C₄-alkoxy, optionally substituted heteroaryl-C₁-C₄-alkoxy, and optionally substituted heteroaryl-; and

R₁₀ and R₁₁ are independently hydrogen, optionally substituted C₁-C₈ alkyl-, optionally substituted aryl-, or optionally substituted aryl-C₁-C₄-alkyl-. See, e.g., PCT/US03/14787, which is incorporated herein by reference.

According to some embodiments, R₉ is phenyl substituted with C₁-C₄-alkyl-, C₁-C₄-alkoxy-, and/or halo; phenyl-; benzyl-; thienyl-; or thienyl- substituted with C₁-C₄-alkyl-, C₁-C₄-alkoxy-, and/or halo. More suitably, R₉ is phenyl substituted with one or more halo and/or methyl.

According to some embodiments, R₁₁ is hydrogen and R₁₀ is substituted C₁-C₄ alkyl-. More suitably, R₁₁ is hydrogen and R₁₀ is aminomethyl-, aminoethyl-, aminopropyl-, acetylamino-methyl-, acetylaminoethyl-, benzyloxycarbonylamino-methyl- or benzyloxycarbonylamino-ethyl-.

In some embodiments of Formula I where T and T′ are not both absent, R₃ raken together with R₆ form an optionally substituted imidazolinyl ring of the formula 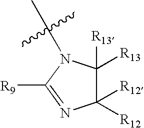
wherein,

R₉ is chosen from hydrogen, optionally substituted C₁-C₈ alkyl-, optionally substituted aryl-, optionally substituted aryl-C₁-C₄-alkyl-, optionally substituted heteroaryl-, optionally substituted heteroaryl-C₁-C₄-alkyl-; and

R₁₂, R_{12′}, R₁₃, and R_{13′} are independently chosen from hydrogen, optionally substituted C₁-C₈ alkyl-, optionally substituted aryl-, and optionally substituted aryl-C₁-C₄-alkyl-.

In some embodiments, R₉ is methylenedioxyphenyl-; phenyl-; phenyl substituted with C₁-C₄ alkyl-, C₁-C₄ alkoxy-, and/or halo; benzyl-; thienyl substituted with C₁-C₄ alkyl; benzyl; thiophenyl-; or thiophenyl- substituted with C₁-C₄-alkyl-, C₁-C₄-alkoxy-, and/or halo. More suitably, R₉ is methylenedioxyphenyl-; phenyl-; tolyl-; methoxyphenyl-; or halomethylphenyl-.

In some embodiments, R₁₂, R_{12′}, R_{13′}, and R₁₃ are independently hydrogen or optionally substituted C₁-C₄ alkyl-. More suitably, R_{13′} and R₁₃ are hydrogen.

When considering the compounds of Formula I, in some embodiments, R₃ taken together with R₆ form an optionally substituted diazepinone ring of the formula: 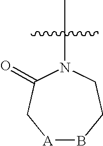
wherein A and B are each independently chosen from C(R₂₀)(R₂₁), N(R₂₂), O, or S, wherein R₂₀ and R₂₁ are each independently selected from H, optionally substituted alkyl, optionally substituted aryl, and optionally substituted heteroaryl; and R₂₂ is H, optionally substituted alkyl, optionally substituted aralkyl, optionally substituted heteroaralkyl, optionally substituted alkylcarbonyl, optionally substituted arylcarbonyl, optionally substituted heteroarylcarbonyl, optionally substituted aralkylcarbonyl, optionally substituted heteroaralkylcarbonyl, optionally substituted alkoxycarbonyl, optionally substituted aryloxycarbonyl, optionally substituted heteroaryloxycarbonyl, optionally substituted aralkyloxycarbonyl, or optionally substituted heteroaralkyloxycarbonyl. In some embodiments, the diazepinone ring is further substituted with one or more of the following groups: optionally substituted alkyl, optionally substituted aryl, optionally substituted aralkyl, optionally substituted heteroaryl, and optionally substituted heteroaralkyl.

In some embodiments of the compounds of Formula I, one of A or B is C(R₂₀)(R₂₁), wherein R₂₀ and R₂₁ are each independently selected from H or C₁-C₄ alkyl, and the other of A or B is N(R₂₂), where R₂₂ is H, C₁-C₄ alkyl, optionally substituted aralkyl, optionally substituted heteroaralkyl, C₁-C₆ alkylcarbonyl, optionally substituted arylcarbonyl, optionally substituted heteroarylcarbonyl, optionally substituted aralkylcarbonyl, optionally substituted heteroaralkylcarbonyl, C₁-C₆ alkoxycarbonyl, optionally substituted aryloxycarbonyl, optionally substituted heteroaryloxycarbonyl, optionally substituted aralkyloxycarbonyl, or optionally substituted heteroaralkyloxycarbonyl, where the optionally substituted aryl or heteroaryl groups or moieties are unsubstituted or substituted with one or more substituents selected from C₁-C₄ alkyl, C₁-C₄ haloalkyl, C₁-C₄ alkoxy, C₁-C₄ haloalkoxy, amino, C₁-C₄ alkylamino, di-C₁-C₄ alkylamino, carboxy, C₁-C₄ alkylcarbonyloxy, C₁-C₄ alkoxycarbonyl, carboxamido, C₁-C₄ alkylcarboxamido, aminocarbonyl, C₁-C₄ alkylaminocarbonyl, di-C₁-C₄ alkylaminocarbonyl, cyano, C₁-C₄ alkylcarbonyl, halogen, hydroxy, mercapto and nitro. In some embodiments, A is C(R₂₀)(R₂₁), wherein R₂₀ and R₂₁ are each H or C₁-C₄ alkyl, and B is N(R₂₂), where R₂₂ is H, C₁-C₄ alkyl, aralkyl, heteroaralkyl, C₁-C₆ alkylcarbonyl, arylcarbonyl, or heteroarylcarbonyl. In some embodiments of the compounds of Formula I, A is CH₂, and B is N(R₂₂), where R₂₂ is H, methyl, benzyl or acetyl (—C(O)methyl). See, e.g., WO 2004/055008, which is incorporated herein by reference for all purposes.

n some embodiments of Formula I, R₃ taken together with R₆ form an optionally substituted piperazine- or diazepam of the formula: 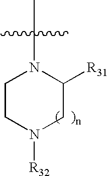
wherein R₃₁ and R₃₂ are independently chosen from hydrogen, optionally substituted alkyl, optionally substituted aryl, optionally substituted heteroaryl, optionally substituted aralkyl, and optionally substituted heteroaralkyl; and n is 1 or 2. In some embodiments, R₃₁ is aryl (such as phenyl), substituted aryl (such as lower alkyl-, lower alkoxy-, and/or halo-substituted phenyl), aralkyl (such as benzyl and phenylvinyl), heteroaralkyl, substituted aralkyl (such as substituted benzyl and substituted phenylvinyl), or substituted heteroaralkyl; R₃₂ is hydrogen; and n is 1. See, e.g., US 2004-0048853, which is incorporated herein by reference.
R₆

In some embodiments, R₆ is chosen from hydrogen, optionally substituted C₁-C₁₃ alkyl-, optionally substituted aryl-, optionally substituted aryl-C₁-C₄-alkyl-, optionally substituted heteroaryl-C₁-C₄-alkyl-, and optionally substituted heterocyclyl-. In some embodiments, R₆ is hydrogen or optionally substituted C₁-C₁₃ alkyl.

In some embodiments, R₆ is chosen from hydrogen, C₁-C₄ alkyl-, cyclohexyl, phenyl substituted with hydroxy, C₁-C₄ alkoxy, or C₁-C₄ alkyl; benzyl; and R₁₆-alkylene-, wherein R₁₆ is hydroxy, carboxy, (C₁-C₄ alkoxy)carbonyl-, di(C₁-C₄ alkyl)amino-, (C₁-C₄ alkyl)amino-, amino, (C₁-C₄ alkoxy)carbonylamino-, C₁-C₄ alkoxy-, optionally substituted furanyl, or optionally substituted N-heterocyclyl-(such as azetidinyl, morpholinyl, pyridinyl, indolyl, pyrrolidinyl, piperidinyl, or imidazolyl, each of which may be optionally substituted).

In some embodiments, R₆ is selected from optionally substituted lower-alkyl-, cyclohexyl-; phenyl substituted with hydroxy, lower-alkoxy or lower-alkyl-; benzyl-; heteroarylmethyl-; heteroarylethyl-; and heteroarylpropyl-.

In some embodiments, R₆ is chosen from methyl-, ethyl-, propyl-, butyl, cyclohexyl, carboxyethyl, carboxymethyl, methoxyethyl, hydroxyethyl, hydroxypropyl, dimethylaminoethyl, dimethylaminopropyl, diethylaminoethyl, diethylaminopropyl, aminopropyl, methylaminopropyl, 2,2-dimethyl-3-(dimethylamino)propyl-, aminoethyl-, aminobutyl, aminopentyl, aminohexyl, isopropylaminopropyl, diisopropylaminoethyl, 1-methyl-4-(diethylamino)butyl, (t-Boc)aminopropyl, hydroxyphenyl, benzyl, methoxyphenyl, methylmethoxyphenyl, dimethylphenyl, tolyl, ethylphenyl, (oxopyrrolidinyl)propyl, (methoxycarbonyl)ethyl, benzylpiperidinyl, pyridinylethyl, pyridinylmethyl, morpholinylethyl, morpholinylpropyl, piperidinyl, azetidinylmethyl, azetidinylethyl, azetidinylpropyl, pyrrolidinylmethyl, pyrrolidinylethyl, pyrrolidinylpropyl, piperidinylmethyl, piperidinylethyl, imidazolylpropyl, imidazolylethyl, (ethylpyrrolidinyl)methyl, (methylpyrrolidinyl)ethyl, (methylpiperidinyl)propyl, (methylpiperazinyl)propyl, furanylmethyl and indolylethyl-.

More suitably, R₆ is R₁₆-alkylene-, wherein R₁₆ is amino, C₁-C₄ alkylamino-, di(C₁-C₄ alkyl)amino-, C₁-C₄ alkoxy-, hydroxy, or N-heterocyclyl. In some embodiments, R₁₆ is amino. In some embodiments, the alkylene moiety of R₁₆-alkylene- has from 1 to 6 carbon atoms.

In some embodiments, R₆ is aminoethyl, aminopropyl, aminobutyl, aminopentyl, aminohexyl, methylaminoethyl, methylaminopropyl, methylaminobutyl, methylaminopentyl, methylaminohexyl, dimethylaminoethyl, dimethylaminopropyl, dimethylaminobutyl, dimethylaminopentyl, dimethylaminohexyl, ethylaminoethyl, ethylaminopropyl, ethylaminobutyl, ethylaminopentyl, ethylaminohexyl, diethylaminoethyl, diethylaminopropyl, diethylaminobutyyl, diethylaminopentyl, or diethylaminohexyl, and in some embodiments, aminopropyl.

R₃

In some embodiments, R₃ is chosen from optionally substituted C₁-C₁₃ alkyl (such as substituted C₁-C₄ alkyl); optionally substituted aralkyl (such as optionally substituted benzyl or naphthylmethyl-); and optionally substituted heteroaralkyl. In some embodiments, R₃ is benzyl or benzyl substituted with one or more of the following groups: carboxy, alkoxycarbonyl, cyano, halo, C₁-C₄ alkyl-, C₁-C₄ alkoxy, nitro, methylenedioxy, or trifluoromethyl.

R₃ is —C(O)R₇

When considering the compounds of Formula I, in some embodiments, R₃ is —C(O)R₇, and R₇ is selected from optionally substituted C₁-C₈ alkyl, optionally substituted aryl-C₁-C₄-alkyl-, optionally substituted heteroaryl-C₁-C₄-alkyl-, optionally substituted heteroaryl, optionally substituted aryl, R₈O1', and R₁₄—NH—, where R₈ is chosen from optionally substituted C₁-C₈ alkyl and optionally substituted aryl, and R₁₄ is chosen from hydrogen, optionally substituted C₁-C₈ alkyl and optionally substituted aryl.

In some embodiments R₇ is selected from optionally substituted C₁-C₈ alkyl, optionally substituted aryl-C₁-C₄-alkyl-, optionally substituted heteroaryl-C₁-C₄-alkyl-, optionally substituted heteroaryl, and optionally substituted aryl. In some embodiments, R₇ is chosen from

phenyl;

phenyl substituted with one or more of the following substituents: halo; C₁-C₄ alkyl; C₁-C₄ alkyl substituted with hydroxy (e.g., hydroxymethyl); C₁-C₄ alkoxy; C₁-C₄ alkyl substituted with C₁-C₄ alkoxy, nitro, formyl, carboxy, cyano, methylenedioxy, ethylenedioxy, acyl (e.g., acetyl), —N-acyl (e.g., N-acetyl), or trifluoromethyl;

benzyl;

phenoxymethyl-;

halophenoxymethyl-;

phenylvinyl-;

heteroaryl;

heteroaryl- substituted with C₁-C₄ alkyl or C₁-C₄ alkyl substituted with halo (e.g., CF₃);

C₁-C₄ alkyl substituted with C₁-C₄ alkoxy-; and

benzyloxymethyl-.

In some embodiments, when R₇ is not R₁₄NH— or R₈O—, R₇ is chosen from phenyl, halophenyl, dihalophenyl, cyanophenyl, halo(trifluoromethyl)phenyl, hydroxymethylphenyl, methoxymethylphenyl, methoxyphenyl, ethoxyphenyl, carboxyphenyl, formylphenyl, ethylphenyl, tolyl, methylenedioxyphenyl, ethylenedixoyphenyl, methoxychlorophenyl, dihydro-benzodioxinyl, methylhalophenyl, trifluoromethylphenyl, furanyl, C₁-C₄ alkyl substituted furanyl, trifluoromethylfuranyl, C₁-C₄ alkyl substituted trifluoromethylfuranyl, benzofuranyl, thiophenyl, C₁-C₄ alkyl substituted thiophenyl, benzothiophenyl, benzothiadiazolyl, pyridinyl, indolyl, methylpyridinyl, trifluoromethylpyridinyl, pyrrolyl, quinolinyl, picolinyl, pyrazolyl, C₁-C₄ alkyl substituted pyrazolyl, N-methyl pyrazolyl, C₁-C₄ alkyl substituted N-methyl pyrazolyl, C₁-C₄ alkyl substituted pyrazinyl, C₁-C₄ alkyl substituted isoxazolyl, benzoisoxazolyl, morpholinomethyl, methylthiomethyl, methoxymethyl, N-methyl imidazolyl, and imidazolyl. In some embodiments, R₇ is optionally substituted phenyl (such as tolyl, halophenyl, methylhalophenyl, hydroxymethylphenyl, halo(trifluoromethyl)phenyl-, methylenedioxyphenyl, formylphenyl or cyanophenyl).

In some embodiments, when R₇ is R₁₄NH—, R₁₄ is chosen from hydrogen, C₁-C₄ alkyl; cyclohexyl; phenyl; and phenyl substituted with halo, C₁-C₄ alkyl, trifluoromethyl, C₁-C₄ alkoxy, or C₁-C₄ alkylthio.

In some embodiments, when R₇ is R₁₄NH—, R₁₄ is hydrogen, isopropyl, butyl, cyclohexyl, phenyl, bromophenyl, dichlorophenyl, methoxyphenyl, ethylphenyl, tolyl, trifluoromethylphenyl or methylthiophenyl.

In some embodiments wherein R₇ is R₈O—, R₈ is chosen from optionally substituted C₁-C₈ alkyl and optionally substituted aryl.

R₃ is SO₂R₇ₐ

In considering compounds of Formula I, in some embodiments, when R₃ is —SO₂R₇ₐ, R₇ₐ is chosen from C₁-C₁₃ alkyl; phenyl; naphthyl; phenyl substituted with halo, C₁-C₄ alkyl, C₁-C₄ alkoxy, cyano, nitro, methylenedioxy, or trifluoromethyl; biphenylyl; and heteroaryl. In some embodiments, R₇ₐ is chosen from naphthyl and phenyl substituted with halo, C₁-C₄ alkyl, C₁-C₄ alkoxy, cyano, nitro, methylenedioxy, and/or trifluoromethyl.

Salt Forms

Compounds of the invention will generally be capable of forming acid addition salts (i.e., will comprise a site that reacts with a pharmaceutically acceptable acid to form an acid addition salt.) The present invention includes pharmaceutically acceptable acid addition salts of the compounds of Formula I. Acid addition salts of the present compounds are prepared in a standard manner in a suitable solvent from the parent compound and an excess of an acid, such as hydrochloric, hydrobromic, sulfuric, phosphoric, acetic, maleic, succinic or methanesulfonic.

The salts and/or solvates of the compounds of Formula I that are not pharmaceutically acceptable may be useful as intermediates in the preparation of pharmaceutically acceptable salts and/or solvates of compounds of Formula I or the compounds of Formula I themselves, and as such form another aspect of the present invention.

In a particular subgenus of compounds of Formula I:

one of T and T′ is absent and the other is optionally substituted alkylene;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl-,

R_{2′} is hydrogen

R₄ is hydrogen, cyano, or optionally substituted methyl;

R₅ is hydrogen, methyl, or cyano; and

R₃ taken together with R₆ and the nitrogen to which they are bound, form an optionally substituted imidazolyl ring.

In a particular subgenus of compounds of Formula I:

T and T′ are independently optionally substituted alkylene;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl-,

R_{2′} is hydrogen

R₄ is hydrogen, cyano, or optionally substituted methyl;

R₅ is hydrogen, methyl, or cyano; and

R₃ taken together with R₆ and the nitrogen to which they are bound, form an optionally substituted imidazolyl ring.

In a particular subgenus of compounds of Formula I:

one of T and T′ is absent and the other is optionally substituted alkylene;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl-,

R_{2′} is hydrogen

R₄ is hydrogen, cyano, or optionally substituted methyl;

R₅ is hydrogen, methyl, or cyano; and

R₃ taken together with R₆ form an optionally substituted imidazolinyl ring.

In a particular subgenus of compounds of Formula I:

T and T′ are independently optionally substituted alkylene;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl-,

R_{2′} is hydrogen

R₄ is hydrogen, cyano, or optionally substituted methyl;

R₅ is hydrogen, methyl, or cyano; and

R₃ taken together with R₆ form an optionally substituted imidazolinyl ring.

In a particular subgenus of compounds of Formula I:

T and T′ are independently optionally substituted alkylene or absent;

R₁ is benzyl, halobenzyl, methylbenzyl, hydroxybenzyl, cyanobenzyl, methoxybenzyl, or naphthalenylmethyl;

R₂ is optionally substituted C₁-C₄alkyl-,

R_{2′} is hydrogen

R₄ is hydrogen, cyano, or optionally substituted methyl;

R₅ is hydrogen, methyl, or cyano; and

R₃ taken together with R₆ form an optionally substituted piperazine- or diazepane ring.

In a particular subgenus of compounds of Formula I:

T and T′ are independently optionally substituted alkylene or absent;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl,

R_{2′} is hydrogen

R₄ is hydrogen, cyano, or optionally substituted methyl;

R₅ is hydrogen, methyl, or cyano; and

R₃ taken together with R₆ form an optionally substituted diazepinone ring.

In a particular subgenus of compounds of Formula I:

one of T and T′ is absent and the other is optionally substituted alkylene;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl-,

R_{2′} is hydrogen

R₄ is hydrogen, cyano, or optionally substituted methyl;

R₅ is hydrogen, methyl, or cyano;

R₆ is R₁₆-alkylene-,

R₁₆ is amino, C₁-C₄ alkylamino-, di(C₁-C₄ alkyl)amino-, C₁-C₄ alkoxy-, hydroxy, or N-heterocyclyl;

R₃ is —C(O)R₇; and

R₇ is optionally substituted phenyl (such as tolyl, halophenyl, methylhalophenyl, hydroxymethylphenyl, halo(trifluoromethyl)phenyl-, methylenedioxyphenyl, formylphenyl or cyanophenyl).

In a particular subgenus of compounds of Formula I:

T and T′ are independently optionally substituted alkylene;

R₁ is benzyl, halobenzyl, methylbenzyl, hydroxybenzyl, cyanobenzyl, methoxybenzyl, or naphthalenylmethyl;

R₂ is optionally substituted C₁-C₄ alkyl-,

R_{2′} is hydrogen

R₄ is hydrogen, cyano, or optionally substituted methyl;

R₅ is hydrogen, methyl, or cyano;

R₆ is R₁₆-alkylene-, R₁₆ is amino, C₁-C₄ alkylamino-, di(C₁-C₄ alkyl)amino-, C₁-C₄ alkoxy-, hydroxy, or N-heterocyclyl;

R₃ is —C(O)R₇; and

R₇ is optionally substituted phenyl (such as tolyl, halophenyl, methylhalophenyl, hydroxymethylphenyl, halo(trifluoromethyl)phenyl-, methylenedioxyphenyl, formylphenyl or cyanophenyl).

In a particular subgenus of compounds of Formula I:

T and T′ are independently optionally lower alkylene or absent;

R₁ is benzyl, halobenzyl, methylbenzyl, hydroxybenzyl, cyanobenzyl, methoxybenzyl, or naphthalenylmethyl;

R_{2′} is hydrogen

R₄ is hydrogen, cyano, or optionally substituted methyl;

R₅ is hydrogen, methyl, or cyano;

R₆ is R₁₆-alkylene-,

R₁₆ is amino, C₁-C₄ alkylamino-, di(C₁-C₄ alkyl)amino-, C₁-C₄ alkoxy-, hydroxy, or N-heterocyclyl; and

R₆ taken together with R₂ form an optionally substituted 5- to 12-membered nitrogen-containing heterocycle, which optionally incorporates from one to two additional heteroatoms, selected from N, O, and S in the heterocycle ring.

Particular compounds include:
5,6-dimethyl-2-{1-[2-(4-methylphenyl)-1-piperazinyl]propyl}-3-(phenylmethyl)-4(3H)-pyrimidinone; and
5-methyl-2-[2-methyl-1-(7-oxohexahydro-1H-1,4-diazepin-1-yl)propyl]-6-oxo-1-(phenylmethyl)-1,6-dihydro-4-pyrimidinecarbonitrile.

Once made, the compounds of the invention find use in a variety of applications involving alteration of mitosis. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In some embodiments, the compounds of the invention are used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest and monopolar spindle formation.

The compounds of the invention are useful to bind to, and/or inhibit the activity of, a mitotic kinesin, KSP. In some embodiments, the KSP is human KSP, although the compounds may be used to bind to or inhibit the activity of KSP kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See U.S. Pat. No. 6,437,115, hereby incorporated by reference in its entirety. The compounds of the invention have been shown to have specificity for KSP. However, the present invention includes the use of the compounds to bind to or modulate other mitotic kinesins.

The compounds of the invention are used to treat cellular proliferation diseases. Such disease states which can be treated by the compounds, compositions and methods provided herein include, but are not limited to, cancer (further discussed below), autoimmune disease, fungal disorders, arthritis, graft rejection, inflammatory bowel disease, cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in some embodiments, the invention herein includes application to cells or individuals afflicted or subject to impending affliction with any one of these disorders or states.

The compounds, compositions and methods provided herein are useful for the treatment of cancer including solid tumors such as skin, breast, brain, cervical carcinomas, testicular carcinomas, etc. In some embodiments, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's. lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

For assay of KSP-modulating activity, generally either KSP or a compound according to the invention is non-diffusably bound to an insoluble support having isolated sample receiving areas (e.g., a microtiter plate, an array, etc.). The insoluble support may be made of any composition to which the sample can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are convenient because a large number of assays can be carried out simultaneously, using small amounts of reagents and samples. The particular manner of binding of the sample is not crucial so long as it is compatible with the reagents and overall methods of the invention, maintains the activity of the sample and is nondiffusable. Particular methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the sample, excess unbound material is removed by washing. The sample receiving areas may then be blocked through incubation with bovine serum albumin (BSA), casein or other innocuous protein or other moiety.

The compounds of the invention may be used on their own to inhibit the activity of a mitotic kinesin, such as KSP. In some embodiments, a compound of the invention is combined with KSP and the activity of KSP is assayed. Kinesin (including KSP) activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, AnaL Biochem. 242 (1):20-5; Gittes et al., 1996, Biophys. J. 70(1): 418-29; Shirakawa et al., 1995, J. Exp. BioL 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Suitably, solution based assays are utilized. U.S. Pat. No. 6,410,254, hereby incorporated by reference in its entirety, describes such assays. Alternatively, conventional methods are used. For example, Pᵢ release from kinesin can be quantified. In some embodiments, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-1 00). To perform the assay, 10 μL of the reaction mixture is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of agents and are well known to those skilled in the art. In some embodiments, ATPase assays of kinesin are performed in the absence of microtubules. In some embodiments, the ATPase assays are performed in the presence of microtubules. Different types of agents can be detected in the above assays. In some embodiments, the effect of an agent is independent of the concentration of microtubules and ATP. In some embodiments, the effect of the agents on kinesin ATPase can be decreased by increasing the concentrations of ATP, microtubules or both. In some embodiments, the effect of the agent is increased by increasing concentrations of ATP, microtubules or both.

Compounds that inhibit the biochemical activity of KSP in vitro may then be screened in vivo. In vivo screening methods include assays of cell cycle distribution, cell viability, or the presence, morphology, activity, distribution, or number of mitotic spindles. Methods for monitoring cell cycle distribution of a cell population, for example, by flow cytometry, are well known to those skilled in the art, as are methods for determining cell viability. See for example, U.S. Pat. No. 6,437,115, hereby incorporated by reference in its entirety. Microscopic methods for monitoring spindle formation and malformation are well known to those of skill in the art (see, e.g., Whitehead and Rattner (1998), J. Cell Sci. 111:2551-61; Galgio et al, (1996) J. Cell Biol., 135:399-414), each incorporated herein by reference in its entirety.

The compounds of the invention inhibit the KSP kinesin. One measure of inhibition is IC₅₀, defined as the concentration of the compound at which the activity of KSP is decreased by fifty percent relative to a control. In some embodiments, the compounds have IC₅₀'s of less than about 1 mM. In some embodiments, the compounds have IC₅₀'s of less than about 100 μM. In some embodiments, the compounds have IC₅₀'s of less than about 10 μM. In some embodiments, the compounds have IC₅₀'s of less than about 1 μM. In some embodiments, the compounds have IC₅₀'s of less than about 100 nM. In some embodiments, the compounds have IC₅₀'s of less than about 10 nM. Measurement of IC₅₀ is done using an ATPase assay such as described herein.

Another measure of inhibition is Kᵢ. For compounds with IC₅₀'s less than 1 μM, the Kᵢ or K_{d} is defined as the dissociation rate constant for the interaction of the compounds described herein with KSP. In some embodiments, the compounds have Kᵢ's of less than about 100 μM. In some embodiments, the compounds have Kᵢ's of less than about 10 μM. In some embodiments, the compounds have Kᵢ's of less than about 1 μM. In some embodiments, the compounds have Kᵢ's of less than about 100 nM. In some embodiments, the compounds have Kᵢ's of less than about 10 nM.

The Kᵢ for a compound is determined from the IC₅₀ based on three assumptions and the Michaelis-Menten equation. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fit to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. In some embodiments, the compounds have GI₅₀'s of less than about 1 mM. In some embodiments, the compounds have a GI₅₀ of less than about 20 μM. In some embodiments, the compounds have a GI₅₀ of less than about 10 μM. In some embodiments, the compounds have a GI₅₀ of less than about 1 μM. In some embodiments, the compounds have a GI₅₀ of less than about 100 nM more so. In some embodiments, the compounds have a GI₅₀ of less than about 10 nM. Measurement of GI₅₀ is done using a cell proliferation assay such as described herein. Compounds of this class were found to inhibit cell proliferation.

In vitro potency of small molecule inhibitors is determined, for example, by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete cell kill).

Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, have potential clinical usefulness.

To employ the compounds of the invention in a method of screening for compounds that bind to KSP kinesin, the KSP is bound to a support, and a compound of the invention is added to the assay. Alternatively, the compound of the invention is bound to the support and KSP is added. Classes of compounds among which novel binding agents may be sought include specific antibodies, non-natural binding agents identified in screens of chemical libraries, peptide analogs, etc. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the compound of the invention to KSP may be done in a number of ways. In some embodiments, the compound is labeled, for example, with a fluorescent or radioactive moiety, and binding is determined directly. For example, this may be done by attaching all or a portion of KSP to a solid support, adding a labeled test compound (for example a compound of the invention in which at least one atom has been replaced by a detectable isotope), washing off excess reagent, and determining whether the amount of the label is that present on the solid support.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the antimitotic agents.

The compounds of the invention may also be used as competitors to screen for additional drug candidates. “Candidate agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, some embodiments exclude molecules already known to bind to that particular protein, for example, polymer structures such as microtubules, and energy sources such as ATP. Some embodiments of assays herein include candidate agents which do not bind the cellular proliferation protein in its endogenous native state termed herein as “exogenous” agents. In some embodiments, exogenous agents further exclude antibodies to KSP.

Candidate agents can encompass numerous chemical classes, though typically they are organic molecules, such as small organic compounds having a molecular weight of more than 100 and less than about 2,500 daltons. Candidate agents comprise finctional groups necessary for structural interaction with proteins, particularly hydrogen bonding and lipophilic binding, and typically include at least an amine, carbonyl-, hydroxy, ether, or carboxyl group, often, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, and/or amidification to produce structural analogs.

Competitive screening assays may be done by combining KSP and a drug candidate in a first sample. A second sample comprises a compound of the present invention, KSP and a drug candidate. This may be performed in either the presence or absence of microtubules. The binding of the drug candidate is determined for both samples, and a change, or difference in binding between the two samples indicates the presence of a drug candidate capable of binding to KSP and potentially inhibiting its activity. That is, if the binding of the drug candidate is different in the second sample relative to the first sample, the drug candidate is capable of binding to KSP.

In some embodiments, the binding of the candidate agent to KSP is determined through the use of competitive binding assays. In some embodiments, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In some embodiments, the candidate agent is labeled. Either the candidate agent, or the competitor, or both, is added first to KSP for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In some embodiments, the competitor is added first, followed by the candidate agent. Displacement of the competitor is an indication the candidate agent is binding to KSP and thus is capable of binding to, and potentially inhibiting, the activity of KSP. In some embodiments, either component can be labeled. Thus, for example, if the competitor is labeled, the presence of label in the wash solution indicates displacement by the agent. Alternatively, if the candidate agent is labeled, the presence of the label on the support indicates displacement.

In some embodiments, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

Inhibition is tested by screening for candidate agents capable of inhibiting the activity of KSP comprising the steps of combining a candidate agent with KSP, as above, and determining an alteration in the biological activity of KSP. In some embodiments, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native KSP, but cannot bind to modified KSP.

Positive controls and negative controls may be used in the assays. Suitably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Accordingly, the compounds of the invention are administered to cells. By “administered” herein is meant administration of a therapeutically effective dose of a compound of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

A “patient” for the purposes of the present invention includes both humans and other animals, such as mammals, and other organisms. Thus the methods are applicable to both human therapy and veterinary applications. In some embodiments, the patient is a mammal. In some embodiments, the patient is human.

Compounds of the invention having the desired pharmacological activity may be administered, for example, as a pharmaceutically acceptable composition comprising an pharmaceutical excipient, to a patient, as described herein. Depending upon the manner of introduction, the compounds may be formulated in a variety of ways as discussed below. The concentration of therapeutically active compound in the formulation may vary from about 0.1-100 wt. %.

The agents may be administered alone or in combination with other treatments, i.e., radiation, or other chemotherapeutic agents such as the taxane class of agents that appear to act on microtubule formation, vinca alkaloids, or the camptothecin class of topoisomerase I inhibitors. When used, other chemotherapeutic agents may be administered before, concurrently, or after administration of a compound of the present invention. In one aspect of the invention, a compound of the present invention is co-administered with one or more other chemotherapeutic agents. By “co-administer” it is meant that the present compounds are administered to a patient such that the present compounds as well as the co-administered compound may be found in the patient's bloodstream at the same time, regardless when the compounds are actually administered, including simultaneously.

The administration of the compounds and compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the compound or composition may be directly applied as a solution or spray.

Pharmaceutical dosage forms include a compound of formula I or a pharmaceutically acceptable salt, solvate, or solvate of a salt thereof, and one or more pharmaceutical excipients. As is known in the art, pharmaceutical excipients are secondary ingredients which function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995), each of which is incorporated herein by reference for all purposes.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colors, flavors, sweetening agents, polymers, waxes or other solubility-retarding materials.

Compositions for intravenous administration will generally comprise intravenous fluids, i.e., sterile solutions of simple chemicals such as sugars, amino acids or electrolytes, which can be easily carried by the circulatory system and assimilated. Such fluids are prepared with water for injection USP.

Fluids used commonly for intravenous (IV) use are disclosed in Remington, the Science and Practice of Pharmacy [full citation previously provided], and include:

alcohol (e.g., in dextrose and water (“D/W”) [e.g., 5% dextrose] or dextrose and water [e.g., 5% dextrose] in normal saline solution (“NSS”); e.g. 5% alcohol);

synthetic amino acid such as Aminosyn, FreAmine, Travasol, e.g., 3.5 or 7; 8.5; 3.5, 5.5 or 8.5 % respectively;

ammonium chloride e.g., 2.14%;

dextran 40, in NSS e.g., 10% or in D5/W e.g., 10%;

dextran 70, in NSS e.g., 6% or in D5/W e.g., 6%;

dextrose (glucose, D5/W) e.g., 2.5-50%;

dextrose and sodium chloride e.g., 5-20% dextrose and 0.22-0.9% NaCl;

lactated Ringer's (Hartmann's) e.g., NaCl 0.6%, KCl 0.03%, CaCl₂ 0.02%;

lactate 0.3%;

mannitol e.g., 5%, optionally in combination with dextrose e.g., 10% or NaCl e.g., 15 or 20%;

multiple electrolyte solutions with varying combinations of electrolytes, dextrose, fructose, invert sugar Ringer's e.g., NaCl 0.86%, KCl 0.03%, CaCl₂ 0.033%;

sodium bicarbonate e.g., 5%;

sodium chloride e.g., 0.45, 0.9, 3, or 5%;

sodium lactate e.g., 1/6 M; and

sterile water for injection

The pH of such fluids may vary, and will typically be from 3.5 to 8 such as known in the art.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All publications, including but not limited to patents and patent applications, cited in this specification are herein incorporated by reference as if each individual publication were specifically and individually indicated to be incorporated by reference herein as though fully set forth.

### EXAMPLES

All anhydrous solvents were purchased from Aldrich Chemical Company in SureSeal® containers.

### Example 1

### 5,6-Dimethyl-2-{1-[2-(4-methylphenyl)-1-piperazinyl]propyl}-3-(phenylmethyl)4(3H)-pyrimidinone

### a). 5,6-Dimethyl-3-(phenylmethyl)-2-propyl-4(3H)-pyrimidinone

5,6-Dimethyl-2-propyl-4(1H)-pyrimidinone (Abstracts of the Journal of the Chemical Society, 5642-59, 1963) is treated with lithium hydride (1.2 equivalents) in dry dioxan at room temperature. When effervesence ceases, a solution of benzyltosylate (1.2 equivalents) in dry dioxan is added and the resulting mixture heated under reflux for 16 h. Water is carefully added and the resulting mixture partitioned between water and ethyl acetate. The organic layer is separated, dried over magnesium sulfate and evaporated under reduced pressure. The residue is purified by chromatography and used in the following steps.

### b). 2-(1-Bromopropyl)-5,6-dimethyl-3-(phenylmethyl)-4(3H)-pyrimidinone

A solution of 5,6-dimethyl-3-(phenylmethyl)-2-propyl-4(3H)-pyrimidinone in glacial acetic acid is treated with sodium acetate (1.2 equivalents) and a solution of bromine in acetic acid (1.1 equivalents). After 1-2 h, the mixture is diluted with water and stirred for an additional 2 h. The precipitated product is isolated, washed well with water and dried.

### c). 5,6-Dimethyl-2-{1-[2-(4-methylphenyl)-1-piperazinyl]propyl}-3-(phenylmethyl)-4(3H)-pyrimidinone

A mixture of 2-(1-bromopropyl)-5,6-dimethyl-3-(phenylmethyl)-4(3H)-pyrimidinone and 1,1-dimethylethyl 3-(4-methylphenyl)-1-piperazinecarboxylate (prepared from 2-(4-methylphenyl)piperazine [EP431991, DE 27184511 and BOC anhydride) is heated under reflux in ethanol until the bromide is consumed. The mixture is concentrated in vacuo and the residue taken up in dichloromethane. Washing with aq. NaOH followed by drying and evaporation of the organic layers gives the crude product which is purified by chromatography. This material is dissolved in dichloromethane and treated with excess trifluoroacetic acid for 3 h. The reaction mixture is evaporated and the residue redissolved in dichloromethane. The solution is washed with aq. NaHCO₃, dried (MgSO4) and concentrated to allow isolation and purification of the desired product.

### Example 2

### 5-Methyl-2-[2-methyl-1-(7-oxohexahydro-1H-1,4-diazepin-1-yl)propyl]-6-oxo-1-(phenylmethyl)-1,6-dihydro-4-pyrimidinecarbonitrile

### a) Boc-D-Valine amide

To a stirred solution of Boc-D-Valine (25 g, 115 mMol) in THF (300 mL) at 0° C. was added N-Methylmorpholine (15 mL, 136 mMol) followed by the dropwise addition of isoButyl chloroformate (18 mL, 139 mMol) over 5 minutes. The reaction was stirred at 0° C. for 30 minutes after which a solution of 30 wt. % NH₄OH (50 mL, 385 mMol) was quickly poured into the reaction. (Vigorous gas evolution was seen which subsided after a few minutes.) The reaction was allowed to warm to RT and stirred for 4 h. The reaction was concentrated under vacuum on the rotoevaporator to a volume which precipitated most of the product. The thick white slurry was diluted with an equal volume of water, filtered, rinsed with water, pressed dry then dried under vacuum to give the title compound (22.56 g, 91%) as a white solid: ¹H NMR (400 MHz, CDCl₃) δ6.17 (br s, 1 H), 5.75 (br s, 1 H), 5.12 (d, 1 H), 4.00 (app. t, 1 H), 2.13 (m, 1 H), 1.44 (s, 9 H), 0.99 (d, 3 H), 0.94 (d, 3 H).

### b) [(R)-1-(N-Benzyl-carbamimidoyl)-2-methyl-propyl]-carbamic acid t-butyl ester

To a stirred solution of Boc-D-Valine amide (10 g, 46 mMol) in CH₂Cl₂ (200 mL) was added triethyloxonium hexafluorophosphate (13.0 g, 48 mMol). (The reaction started out as a suspension which gradually cleared.) The reaction was stirred for 48 h at RT, poured into a separatory funnel, washed with 1 N Na₂CO₃, dried (Na₂SO₄), filtered and concentrated under vacuum. To the remaining oil was added Benzylamine (5.0 mL, 23 mMol) and EtOH (20 mL). The reaction was stirred at 60° C. for 24 h. After cooling to RT the reaction was evaporated under vacuum. The title compound (14.16 g, >95% pure by LCMS) was obtained without purification as a pale yellow oil which eventually solidified to a waxy solid: MS (ES) m/e 306.4 (M+H)⁺.

### c) [1-(3-Benzyl-5-methyl-6-hydroxy4-oxo-3,4-dihydro-pyrimidin-2-yl)-2-methyl-propyl]-carbamic acid-t-butyl ester

To a stirred solution of [(R)-1-(N-Benzyl-carbamimidoyl)-2-methyl-propyl]-carbamic acid t-butyl ester (16.37 g, 53.6 mMol) in CH₂Cl₂ (150 mL) with cooling at 0° C. was added Et₃N (9 mL, 64.3 mMol) followed by 2-chlorocarbonyl-propionic acid ethyl ester (10 g, 60.8 mMol) dropwise over 15 minutes. The reaction was allowed to warm to RT and stirred for 4 h, poured into a separatory funnel, washed with water, brine, dried (Na₂SO₄), and evaporated to dryness under vacuum. The unpurified acylamidine (˜90% pure by LCMS, MS (ES) m/e (M+H)⁺ 434.4) was taken up in DMF (150 mL) and heated to 100° C. with stirring for 18 h. The reaction was concentrated under vacuum and purified by flash chromatography (90:10:1, CH₂Cl₂:EtOAc:HOAc) then (30:70:1, EtOAc:hexane:HOAc) to give the title compound (8.42 g, 41%) as an off-white solid: MS (ES) m/e 388.2 (M +H)⁺.

### d) [1-(3-Benzyl-5-methyl-6-cyano4-oxo-3,4-dihydro-pyrimidin-2-yl)-2-methyl-propyl]-carbamic acid-t-butyl ester

To a stirred solution of [1-(3-benzyl-5-methyl-6-hydroxy-4-oxo-3,4-dihydro-pyrimidin-2-yl)-2-methyl-propyl]-carbamic acid-t-butyl ester (8.42 g, 21.7 mMol) in DMF (150 mL) was added portionwise a 60% dispersion of NaH in mineral oil (0.95 g, 24 mMol). After stirring for 15 minutes at RT, N-phenyltrifluoromethanesulfonimide (8.6 g, 24 mMol) was added. The reaction was stirred at RT for 18 h, concentrated under vacuum, taken up in EtOAc, washed with sat. NH₄Cl, dried (MgSO4), filtered and evaporated under vacuum. Purification by flash chromatography (step gradient of 0 to 5 % EtOAc in CH₂Cl₂) then (10% EtOAc/hexane) gave the semi-purified triflate (11.70 g), (MS (ES) m/e 520.2 (M+H)⁺, contained 23% PhNHSO₂CF₃ by LCMS) as a white solid. To the crude triflate with stirring in DMF (150 mL) was added Zn(CN)₂ (2.6 g, 22.2 mMol) and (PPh₃)₄Pd (2.6 g, 2.3 mMol). The reaction was heated under Ar at 90° C. for 4 h, cooled to RT, and evaporated under vacuum. Purification by flash chromatography (step gradient of 0 to 5 % EtOAc in CH₂Cl₂) gave the title compound (7.11 g, 82%) as a white solid: MS (ES) m/e 520.2 (M+H)⁺.

### e) 2-(1-Amino-2-methylpropyl)-5-methyl-6-oxo-1-(phenylmethyl)-1,6-dihydro4-pyrimidinecarbonitrile

To a solution of 1-(3-benzyl-5-methyl-6-cyano-4-oxo-3,4-dihydro-pyrimidin-2-yl)-2-methyl-propyl]-carbamic acid-t-butyl ester (0.12 mMol) in dichloromethane (10 mL) is added trifluoroacetic acid (10 mL) at room temperature. The resulting solution is stirred at room temperature for one hour and then concentrated under reduced pressure. The residue is dried under high vacuum and dissolved in ethyl acetate (25 mL). It is neutralized with saturated aqueous sodium bicarbonate solution (25 mL), and the aqueous phase is extracted with ethyl acetate (3×25 mL). The combined organic layers are dried over sodium sulfate. After evaporation of solvents, the residue is purified via flash column chromatography (NH₄OH/MeOH/DCM 0.1:1:10 as eluent). The desired product is isolated and characterised.

### f) (2-Oxo-ethyl)-carbamic acid tert-butyl ester

To a stirred solution of oxalyl chloride (1.92 mL, 22 mMol) in CH₂Cl₂ (40 mL) was added dropwise DMSO (3.12 mL, 44 mMol) at −78° C. After 15 min., a solution of (2-hydroxy-ethyl)-carbamic acid tert-butyl ester (3.22 g, 20 mMol) in CH₂Cl₂ (20 mL) was added. After another 45 min., Et₃N (13.9 mL, 100 mMol) was added. The reaction mixture was then warmed to room temperature, diluted with CH₂Cl₂ (100 mL), washed with water, 10% HCl, brine, dried and concentrated. Purification by flash chromatography on silica gel (10-15% EtOAc in hexane) gave the title compound (600 mg) as a clear oil: ¹H NMR (400 MHz, CDCl₃) δ9.58 (S, 1 H), 5.16 (brs, 1 H), 4.02 (s, 2 H), 1.46 (s, 9 H).

### g) 1,1-Dimethylethyl[2-({1-[4-cyano-5-methyl-6-oxo-1-(phenylmethyl)-1,6-dihydro-2-pyrimidinyl]-2-methylpropyl}amino)ethyl]carbamate

Sodium triacetoxyborohydride (1.5 equivalents) is added to a solution 2-(1-amino-2-methylpropyl)-5-methyl-6-oxo-1-(phenylmethyl)-1,6-dihydro-4-pyrimidinecarbonitrile and (2-oxo-ethyl)-carbamic acid tert-butyl ester (1.5 equivalents) in CH₂Cl₂. The resulting mixture is stirred at room temperature overnight. The reaction is diluted with CH₂Cl₂, washed with brine, dried and concentrated under vacuum. Purification by flash chromatography on silica gel leads to the title compound.

### h) 1,1-Dimethylethyl[2-(acryloyl{1-[4-cyano-5-methyl-6-oxo-1-(phenylmethyl)-1,6-dihydro-2-pyrimidinyl]-2-methylpropyl}amino)ethyl]carbamate

Acryloyl chloride (1.6 equivalents) is added to 1,1-dimethylethyl [2-({1-[4-cyano-5-methyl-6-oxo-1-(phenylmethyl)-1,6-dihydro-2-pyrimidinyl]-2-methylpropyl}amino)ethyl]carbamate and Et₃N (2 equivalents) in CH₂Cl₂. The resulting mixture is stirred at room temperature overnight. The reaction mixture is diluted with CH₂Cl₂, washed with brine, dried and concentrated under vacuum. Purification by flash chromatography on silica gel gives the title compound.

### i) N-(2-aminoethyl)-N-{1-[4-cyano-5-methyl-6-oxo-1-(phenylmethyl)-1,6-dihydro-2-pyrimidinyl]-2-methylpropyl}-2-propenamide

1,1-Dimethylethyl[2-(acryloyl {1-[4-cyano-5-methyl-6-oxo-1-(phenylmethyl)1,6-dihydro-2-pyrimidinyl]-2-methylpropyl}amino)ethyl]carbamate is treated with 50% TFA in CH₂Cl₂ at room temperature. After 2 h the mixture is concentrated under vacuum, redissolved in CH₂Cl₂, washed with 10% NaHCO₃, brine, dried and concentrated to give the title compound.

### j) 5-Methyl-2-[2-methyl-1-(7-oxohexahydro-1H-1,4-diazepin-1-yl)propyl]-6-oxo-1-(phenylmethyl)-1,6-dihydro4-pyrimidinecarbonitrile

A solution N-(2-aminoethyl)-N-{1-{4-cyano-5-methyl-6-oxo-1-(phenylmethyl)-1,6-dihydro-2-pyrimidinyl]-2-methylpropyl}-2-propenarnide in MeOH is refluxed under argon overnight. The reaction mixture is concentrated and the residue purified by flash chromatography on silica gel to give the title compound.

### Example 3

### Inhibition of Cellular Viability in Tumor Cell Lines Treated with KSP Inhibitors

Materials and Solutions:

Cells: SKOV3, Ovarian Cancer (human).

Media: Phenol Red Free RPMI+5% Fetal Bovine Serum+2mM L-glutamine.

Colorimetric Agent for Determining Cell Viability: Promega MTS tetrazolium compound.

Control Compound for max cell kill: Topotecan, 1 μM.

Procedure: Day 1—Cell Plating:

Adherent SKOV3 cells are washed with 10 mL of PBS followed by the addition of 2 mL of 0.25% trypsin and incubation for 5 minutes at 37° C. The cells are rinsed from the flask using 8 mL of media (phenol red-free RPMI+5%FBS) and transferred to fresh flask. Cell concentration is determined using a Coulter counter and the appropriate volume of cells to achieve 1000 cells/100 μL is calculated. 100 μL of media cell suspension (adjusted to 1000 cells/100 μL) is added to all wells of 96-well plates, followed by incubation for 18 to 24 hours at 37° C., 100% humidity, and 5% CO₂ allowing the cells to adhere to the plates.

Procedure: Day 2—Compound Addition:

To one column of the wells of an autoclaved assay block are added an initial 2.5 μL of test compound(s) at 400× the highest desired concentration. 1.25 μL of 400× (400 μM) Topotecan is added to other wells (optical density's from these wells are used to subtract out for background absorbance of dead cells and vehicle). 500 μL of media without DMSO are added to the wells containing test compound, and 250 μL to the Topotecan wells. 250 μL of media+0.5% DMSO is added to all remaining wells, into which the test compound(s) are serially diluted. By row, compound-containing media is replica plated (in duplicate) from the assay block to the corresponding cell plates. The cell plates are incubated for 72 hours at 37° C., 100% humidity, and 5% CO₂.

Procedure: Day 4—MTS Addition and OD Reading:

The plates are removed from the incubator and 40 μl MTS PMS is added to each well. Plates are then incubated for 120 minutes at 37° C., 100% humidity, 5% CO₂, followed by reading the ODs at 490 nm after a 5 second shaking cycle in a ninety-six well spectrophotometer.

Data Analysis

The normalized % of control (absorbance- background) is calculated and an XLfit is used to generate a dose-response curve from which the concentration of compound required to inhibit viability by 50% is determined. The compounds of the present invention show activity when tested by this method as described above.

### Example 4

Enantiomer Separation

In general, the procedures described above can be used to prepare substantially pure or enriched R- or S-enantiomers by selected a starting amino acid of the appropriate R- or S-configuration. More preferred compounds of the invention are those of the R-configuration at the stereogenic center to which R₂ is attached. An R:S mixture can be separated into its constituent pure enantiomers by methods well known to those skilled in the art. These include the formation and separation of diastereomeric derivatives such as those formed by reaction with an optically pure acid such as dibenzoyltartaric acid. Alternatively, separation can be accomplished by chiral chromatography, for example, using the following conditions:

Column: Chiralcel OD 20×250 mm;

Sample loaded ˜100 mg mL¹ in 1:2 ethanol:hexane containing 0.01% isopropylamine;

Chromatography conditions: isocratic elution with 1:2 ethanol:hexane containing 0.01% isopropylamine at a flow rate of 15 mL min^{31 1};

UV detection at 254 nm.

For example, an enriched 3:1 R:S mixture of enantiomers was separated into its pure enantiomers by chiral chromatography with the following conditions: Chiralpak AD, 250×4.6 mm (Diacel Inc.). Sample—22.5 mg/ml in 1:1 i-PrOH:hexanes. Conditions: 40 min at isocratic 50% i-PrOH in Hexanes, (S)-enantiomer elutes at 18.35 min, (R)-enantiomer elutes at 26.87 min. The (R)-enantiomer was significantly more potent than the (S)-enantiomer.

### Example 5

### Monopolar Spindle Formation Following Application of a KSP Inhibitor

Human tumor cells Skov-3 (ovarian) were plated in 96-well plates at densities of 4,000 cells per well, allowed to adhere for 24 hours, and treated with various concentrations of the pyridmidinone derivatives for 24 hours. Cells were fixed in 4% formaldehyde and stained with antitubulin antibodies (subsequently recognized using fluorescently-labeled secondary antibody) and Hoechst dye (which stains DNA).

Visual inspection revealed that the compounds caused cell cycle arrest in the prometaphase stage of mitosis. DNA was condensed and spindle formation had initiated, but arrested cells uniformly displayed monopolar spindles, indicating that there was an inhibition of spindle pole body separation. Microinjection of anti-KSP antibodies also causes mitotic arrest with arrested cells displaying monopolar spindles.

### Example 6

### Inhibition of Cellular Proliferation in Tumor Cell Lines Treated with KSP Inhibitors

Cells were plated in 96-well plates at densities from 1000-2500 cells/well of a 96-well plate and allowed to adhere/grow for 24 hours. They were then treated with various concentrations of drug for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CeIlTiter 96® AQᵤₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this.

A Gi₅₀ was calculated by plotting the concentration of compound in μM vs the percentage of cell growth in treated wells. The Gi₅₀ calculated for the compounds is the estimated concentration at which growth is inhibited by 50% compared to control, i.e., the concentration at which:
100×[(Treated₄₈−T₀)/(Control₄₈−T₀)]=50
wherein Treated₄₈ is the value at 48 hours for the treated cells and Control₄₈ is the value at 48 hours for the control population.

All concentrations of compounds are tested in duplicate and controls are averaged over 12 wells. A very similar 96-well plate layout and Gi₅₀ calculation scheme is used by the National Cancer Institute (see Monks, et al., J. Natl. Cancer Inst. 83:757-766 (1991)). However, the method by which the National Cancer Institute quantitates cell number does not use MTS, but instead employs alternative reagents or methods.

Compounds of Examples 1-13 above inhibited cell proliferation in human ovarian tumor cell lines (SKOV-3).

### Example 7

Calculation of IC₅₀:

Measurement of a compound's IC₅₀ for KSP activity uses an ATPase assay. The following solutions are used: Solution 1 consists of 3 mM phosphoenolpyruvate potassium salt (Sigma P-7127), 2 mM ATP (Sigma A-3377), 1 mM IDTT (Sigma D-9779), 5 μM paclitaxel (Sigma T-7402), 10 ppm antifoam 289 (Sigma A-8436), 25 mM Pipes/KOH pH 6.8 (Sigma P6757), 2 mM MgCl₂ (VWR JT400301), and 1 mM EGTA (Sigma E3889). Solution 2 consists of 1 mM NADH (Sigma N8129), 0.2 mg/ml BSA (Sigma A7906), pyruvate kinase 7U/mL, L-lactate dehydrogenase 10 U/ml (Sigma P0294), 100 nM KSP motor domain, 50 μg/mL microtubules, 1 mM DTT (Sigma D9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT4003-01), and 1 mM EGTA (Sigma E3889). Serial dilutions (8-12 two-fold dilutions) of the compound are made in a 96-well microtiter plate (Coming Costar 3695) using Solution 1. Following serial dilution each well has 50 μL of Solution 1. The reaction is started by adding 50 μL of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a finction of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x is the compound concentration.

1. At least one chemical entity chosen from compounds of Formula I 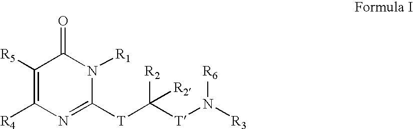
and pharmaceutically acceptable salts, solvates, crystal forms, diastereomers, and prodrugs thereof wherein:
T and T′ are independently optionally substituted lower alkylene or absent;
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R₂ are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring;
R₃ is selected from hydrogen, optionally substituted alkyl-, optionally substituted aryl-, optionally substituted aralkyl-, optionally substituted heteroaryl-, optionally substituted heteroaralkyl-, —(CO)R₇, and —SO₂R₇ₐ;
or R₃ taken together with R₆, and the nitrogen to which they are bound, form an optionally substituted 5- to 12-membered nitrogen-containing heterocycle, which optionally incorporates from one to two additional heteroatoms, selected from N, O, and S in the heterocycle ring;
or R₆ taken together with R₂ form an optionally substituted 5- to 1 2-membered nitrogen-containing heterocycle, which optionally incorporates from one to two additional heteroatoms, selected from N, O, and S in the heterocycle ring;
R₄ and R₅ are independently chosen from hydrogen, optionally substituted alkyl-, optionally substituted alkoxy, acyl, halogen, hydroxy, nitro, cyano, alkylsulfonyl-, alkylsulfanyl-, aminocarbonyl-, optionally substituted amino, optionally substituted aryl-, optionally substituted aralkyl-, optionally substituted heteroaralkyl and optionally substituted heteroaryl-;
R₆ is chosen from hydrogen, optionally substituted alkyl-, optionally substituted aryl-, optionally substituted aralkyl-, optionally substituted heteroaralkyl-, and optionally substituted heterocyclyl-;
R₇ is chosen from hydrogen, optionally substituted alkyl-, optionally substituted aryl-, optionally substituted aralkyl-, optionally substituted heteroaryl-, optionally substituted heteroaralkyl-, R₈O— and R₁₄—NH—;
R₇ₐ is chosen from optionally substituted alkyl-, optionally substituted aryl-, optionally substituted aralkyl-, optionally substituted heteroaryl-, optionally substituted heteroaralkyl-, and R₁₄—NH—;
R₈ is chosen from optionally substituted alkyl-, optionally substituted aryl-, optionally substituted aralkyl-, optionally substituted heteroaryl-, and optionally substituted heteroaralkyl-; and
R₁₄ is chosen from hydrogen, optionally substituted alkyl-, optionally substituted aryl-, optionally substituted aralkyl-, optionally substituted heteroaryl-, and optionally substituted heteroaralkyl-
provided that:
at least one the following criteria is met:
T and T′ are not both absent; or
R₆ taken together with R₂ form an optionally substituted 5- to 12-membered nitrogen-containing heterocycle, which optionally incorporates from one to two additional heteroatoms, selected from N, O, and S in the heterocycle ring; or
R₃ taken together with R₆, and the nitrogen to which they are bound, form an optionally substituted 5- to 12-membered nitrogen-containing heterocycle, which optionally incorporates from one to two additional heteroatoms, selected from N, O, and S in the heterocycle ring wherein said heterocycle is not an imidazole or imidazoline ring when T and T′ are both absent.

2. At least one chemical entity of claim 1, wherein R₁ is selected from hydrogen, optionally substituted C₁-C₄ alkyl-, optionally substituted phenyl-C₁-C₄-alkyl-, optionally substituted heteroaryl-C₁-C₄ alkyl, optionally substituted naphthalenylmethyl-, optionally substituted phenyl-, and naphthyl-.

3. At least one chemical entity of claim 1, wherein R₁ is optionally substituted phenyl-C₁-C₄-alkyl-, optionally substituted heteroaryl-C₁-C₄-alkyl-, optionally substituted naphthalenylmethyl-, optionally substituted phenyl, or naphthyl.

4. At least one chemical entity of claim 1, wherein R₁ is naphthyl-, phenyl-, bromophenyl-, chlorophenyl-, methoxyphenyl-, ethoxyphenyl-, tolyl-, dimethylphenyl-, chorofluorophenyl-, methylchlorophenyl-, ethylphenyl-, phenethyl-, benzyl-, halobenzyl-, methylbenzyl-, methoxybenzyl-, cyanobenzyl-, hydroxybenzyl-, dichlorobenzyl-, dimethoxybenzyl-, or naphthalenylmethyl-.

5. At least one chemical entity of claim 1, wherein R₁ is optionally substituted phenyl-C₁-C₄ alkyl or optionally substituted heteroaryl-CI -C₄ alkyl.

6. At least one chemical entity of claim 1, wherein R₁ is benzyl-, halobenzyl, methylbenzyl, hydroxybenzyl-, cyanobenzyl-, methoxybenzyl-, or naphthalenylmethyl-.

7. At least one chemical entity of claim 1, wherein R₁ is benzyl-.

8. At least one chemical entity of claim 1, wherein R₂ is optionally substituted C₁-C₄ alkyl-, and R_{2′} is hydrogen or optionally substituted C₁-C₄ alkyl-.

9. At least one chemical entity of claim 8, wherein R_{2′} is hydrogen and R₂ is optionally substituted C₁-C₄ alkyl-.

10. At least one chemical entity of claim 9, wherein R₂ is chosen from methyl-, ethyl-, propyl, butyl, methylthioethyl-, methylthiomethyl-, aminobutyl-, (CBZ)aminobutyl-, cyclohexylmethyl-, benzyloxymethyl-, methylsulfanylethyl-, methylsulfanylmethyl-, and hydroxymethyl-, and R_{2′} is hydrogen.

11. At least one chemical entity of claim 10, wherein R_{2′} is hydrogen and R₂ is ethyl or propyl.

12. At least one chemical entity of claim 11, wherein R₂ is i-propyl.

13. At least one chemical entity of claim 1, wherein R₂ or R_{2′} is hydrogen and the other is not hydrogen.

14. At least one chemical entity of claim 1, wherein R₂ and R₆ taken together form a 5- to 12-membered ring which optionally incorporates one or two additional heteroatoms, selected from N, O, and S in the heterocycle ring and may optionally be substituted with one or more of the following groups: alkyl, aryl, aralkyl, heteroaryl, substituted alkyl, subtituted aryl, substituted aralkyl, substituted heteroaryl, hydroxy, alkoxy, cyano, optionally substituted amino, and oxo.

15. At least one chemical entity of claim 1, wherein R₄ is hydrogen, acyl, alkoxy, cyano, carboxy, optionally substituted amino, aminocarbonyl, lower-alkyl, lower-alkyl substituted with one or more of the following substituents: halo, lower-alkoxy, or hydroxy, phenyl, or phenyl substituted with one or more of the following substituents: halo, lower-alkoxy, or hydroxy.

16. At least one chemical entity of claim 15, wherein R₄ is hydrogen, cyano, methyl, or methyl substituted with one or more of the following substituents: halo, lower-alkoxy, or hydroxy.

17. At least one chemical entity of claim 1, wherein R₅ is hydrogen, acyl, carboxy, aminocarbonyl, optionally substituted amino, cyano, lower-alkyl, halo, benzyl, piperonyl, naphthyl, furyl, thienyl, indolyl, morpholinyl, phenyl, benzodioxolyl, or phenyl substituted with one or more of the following substituents: optionally substituted amino, aminocarbonyl, cyano, halo, optionally substituted lower-alkyl-, optionally substituted lower-alkoxy, optionally substituted lower-alkyl sulfanyl, hydroxy, or thio.

18. At least one chemical entity of claim 17, wherein R₅ is hydrogen, methyl; ethyl; bromo; carboxy; cyano; phenyl; halophenyl; lower-alkylphenyl; trifluoromethylphenyl; lower-alkoxyphenyl; di(lower-alkoxy)phenyl; polyhalophenyl; halo lower-alkylphenyl; furyl; thienyl; lower-alkylsulfanylphenyl; thiophenyl; aminophenyl; aminocarbonylphenyl; cyanophenyl; di(lower-alkyl)aminophenyl; di(lower-alkyl)phenyl; acetylaminophenyl; amino substituted lower-alkylphenyl; hydroxy substituted lower-alkylphenyl; piperonyl; naphthyl; carbamoyl; lower-alkyl carbamoyl; benzylcarbamoyl; phenylcarbamoyl; methoxymethyl carbamoyl; methoxyethyl carbamoyl; hydroxymethyl carbamoyl; hydroxyethyl carbamoyl; indolyl; morpholinyl; and morpholinocarbonyl.

19. At least one chemical entity of claim 18, wherein R₅ is hydrogen, methyl, or cyano.

20. At least one chemical entity of claim 1, wherein one of T or T′ is absent and the other is optionally substituted alkylene.

21. At least one chemical entity of claim 1, wherein both T and T′ are absent.

22. At least one chemical entity of claim 1 any, wherein R₃ taken together with R₆, and the nitrogen to which they are bound, form an optionally substituted 5- to 12-membered nitrogen-containing heterocycle, which optionally incorporates from one to two additional heteroatoms, selected from N, O, and S, in the heterocycle ring and may optionally be substituted with one or more of the following groups: alkyl, aryl, aralkyl, heteroaryl, substituted alkyl, substituted aryl, substituted aralkyl, substituted heteroaryl, hydroxy, alkoxy, cyano, optionally substituted amino, and oxo.

23. At least one chemical entity of claim 22, wherein T and T′ are not both absent and R₃ taken together with R₆ and the nitrogen to which they are bound, form an optionally optionally substituted imidazolyl ring of the formula: 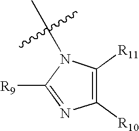
wherein
R₉ is chosen from hydrogen, optionally substituted C₁-C₈ alkyl-, optionally substituted aryl-, optionally substituted aryl-C₁-C₄-alkyl-, optionally substituted heteroaryl-C₁-C₄-alkyl-, optionally substituted aryl-C₁-C₄-alkoxy, optionally substituted heteroaryl-Ci-C₄-alkoxy, and optionally substituted heteroaryl-; and
R₁₀ and R₁₁ are independently hydrogen, optionally substituted C₁-C₈ alkyl-, optionally substituted aryl-, or optionally substituted aryl-C₁-C₄-alkyl-.

24. At least one chemical entity of claim 22, wherein T and T′ are not both absent, and R₃ taken together with R₆ form an optionally substituted imidazolinyl ring of the formula 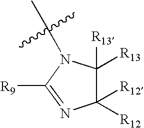
wherein,
R₉ is chosen from hydrogen, optionally substituted C₁-C₈ alkyl-, optionally substituted aryl-, optionally substituted aryl-C₁-C₄-alkyl-, optionally substituted heteroaryl-, optionally substituted heteroaryl-C₁-C₄-alkyl-; and
R₁₂, R_{12′}, R₁₃, and R_{13′} are independently chosen from hydrogen, optionally substituted C₁-C₈ alkyl-, optionally substituted aryl-, and optionally substituted aryl-C₁-C₄-alkyl-.

25. At least one chemical entity of claim 22, wherein R₃ taken together with R₆ form an optionally substituted diazepinone ring of the formula: 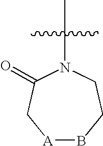
wherein A and B are each independently chosen from C(R₂₀)(R₂₁), N(R₂₂), O, or S, wherein R₂₀ and R₂₁ are each independently selected from H, optionally substituted alkyl, optionally substituted aryl, and optionally substituted heteroaryl; and R₂₂ is H, optionally substituted alkyl, optionally substituted aralkyl, optionally substituted heteroaralkyl, optionally substituted alkylcarbonyl, optionally substituted arylcarbonyl, optionally substituted heteroarylcarbonyl, optionally substituted aralkylcarbonyl, optionally substituted heteroaralkylcarbonyl, optionally substituted alkoxycarbonyl, optionally substituted aryloxycarbonyl, optionally substituted heteroaryloxycarbonyl, optionally substituted aralkyloxycarbonyl, or optionally substituted heteroaralkyloxycarbonyl.

26. At least one chemical entity of claim 22, wherein R₃ taken together with R₆ form an optionally substituted piperazine- or diazepam of the formula: 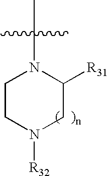
wherein R₃₁ and R₃₂ are independently chosen from hydrogen, optionally substituted alkyl, optionally substituted aryl, optionally substituted heteroaryl, optionally substituted aralkyl, and optionally substituted heteroaralkyl; and n is 1 or 2.

27. At least one chemical entity of claim 1, wherein R₆ is hydrogen or optionally substituted C₁-C₁₃ alkyl.

28. At least one chemical entity of claim 27, wherein R₆ is chosen from hydrogen, C₁-C₄ alkyl-, cyclohexyl, phenyl substituted with hydroxy, C₁-C₄ alkoxy, or C₁-C₄ alkyl; benzyl; and R₁₆-alkylene-, wherein R₁₆ is hydroxy, carboxy, (C₁-C₄ alkoxy)carbonyl-, di(C₁-C₄ alkyl)amino-, (C₁-C₄ alkyl)amino-, amino, (C₁-C₄ alkoxy)carbonylamino-, C₁-C₄ alkoxy-, optionally substituted furanyl, or optionally substituted N-heterocyclyl-.

29. At least one chemical entity of claim 28, wherein R₆ is selected from optionally substituted lower-alkyl-, cyclohexyl-; phenyl substituted with hydroxy, lower-alkoxy or lower-alkyl-; benzyl-; heteroarylmethyl-; heteroarylethyl-; and heteroarylpropyl-.

30. At least one chemical entity of claim 29, wherein R₆ is chosen from methyl-, ethyl-, propyl-, butyl, cyclohexyl, carboxyethyl, carboxymethyl, methoxyethyl, hydroxyethyl, hydroxypropyl, dimethylaminoethyl, dimethylaminopropyl, diethylaminoethyl, diethylaminopropyl, aminopropyl, methylaminopropyl, 2,2-dimethyl-3-(dimethylamino)propyl-, aminoethyl-, aminobutyl, aminopentyl, aminohexyl, isopropylaminopropyl, diisopropylaminoethyl, 1-methyl-4-(diethylamino)butyl, (t-Boc)aminopropyl, hydroxyphenyl, benzyl, methoxyphenyl, methylmethoxyphenyl, dimethylphenyl, tolyl, ethylphenyl, (oxopyrrolidinyl)propyl, (methoxycarbonyl)ethyl, benzylpiperidinyl, pyridinylethyl, pyridinylmethyl, morpholinylethyl, morpholinylpropyl, piperidinyl, azetidinylmethyl, azetidinylethyl, azetidinylpropyl, pyrrolidinylmethyl, pyrrolidinylethyl, pyrrolidinylpropyl, piperidinylmethyl, piperidinylethyl, imidazolylpropyl, imidazolylethyl, (ethylpyrrolidinyl)methyl, (methylpyrrolidinyl)ethyl, (methylpiperidinyl)propyl, (methylpiperazinyl)propyl, furanylmethyl and indolylethyl-.

31. At least one chemical entity of any of claim 1, wherein R₆ is R₁₆-alkylene-, wherein R₁₆ is amino, C₁-C₄ alkylamino-, di(C₁-C₄ alkyl)amino-, C₁-C₄ alkoxy-, hydroxy, or N-heterocyclyl.

32. At least one chemical entity of claim 31, wherein R₁₆ is amino.

33. At least one chemical entity of claim 32, wherein R₆ is aminoethyl, aminopropyl, aminobutyl, aminopentyl, aminohexyl, methylaminoethyl, methylaminopropyl, methylaminobutyl, methylaminopentyl, methylaminohexyl, dimethylaminoethyl, dimethylaminopropyl, dimethylaminobutyl, dimethylaminopentyl, dimethylaminohexyl, ethylaminoethyl, ethylaminopropyl, ethylaminobutyl, ethylaminopentyl, ethylaminohexyl, diethylaminoethyl, diethylaminopropyl, diethylaminobutyyl, diethylaminopentyl, or diethylaminohexyl, and in some embodiments, aminopropyl.

34. At least one chemical entity of claim 1, R₃ is chosen from optionally substituted C₁-C₁₃ alkyl; optionally substituted aralkyl; and optionally substituted heteroaralkyl.

35. At least one chemical entity of claim 1, wherein R₃ is —C(O)R₇ and R₇ is selected from optionally substituted C₁-C₈ alkyl, optionally substituted aryl-C₁-C₄-alkyl-, optionally substituted heteroaryl-C₁-C₄-alkyl-, optionally substituted heteroaryl, optionally substituted aryl, R₈O—, and R₁₄—NH—, where R₈ is chosen from optionally substituted C₁-C₈ alkyl and optionally substituted aryl, and R₁₄ is chosen from hydrogen, optionally substituted C₁-C₈ alkyl and optionally substituted aryl.

36. At least one chemical entity of claim 35, wherein R₃ is —C(O)R₇ and R₇ is selected from optionally substituted C₁-C₈ alkyl, optionally substituted aryl-C₁-C₄-alkyl-, optionally substituted heteroaryl-C -C₄-alkyl-, optionally substituted heteroaryl, and optionally substituted aryl.

37. At least one chemical entity of claim 36, wherein R₃ is —C(O)R₇ and R₇ is chosen from phenyl, halophenyl, dihalophenyl, cyanophenyl, halo(trifluoromethyl)phenyl, hydroxymethylphenyl, methoxymethylphenyl, methoxyphenyl, ethoxyphenyl, carboxyphenyl, formylphenyl, ethylphenyl, tolyl, methylenedioxyphenyl, ethylenedixoyphenyl, methoxychlorophenyl, dihydro-benzodioxinyl, methylhalophenyl, trifluoromethylphenyl, furanyl, C₁-C₄ alkyl substituted furanyl, trifluoromethylfuranyl, C₁-C₄ alkyl substituted trifluoromethylfuranyl, benzofuranyl, thiophenyl, C₁-C₄ alkyl substituted thiophenyl, benzothiophenyl, benzothiadiazolyl, pyridinyl, indolyl, methylpyridinyl, trifluoromethylpyridinyl, pyrrolyl, quinolinyl, picolinyl, pyrazolyl, C₁-C₄ alkyl substituted pyrazolyl, N-methyl pyrazolyl, C₁-C₄ alkyl substituted N-methyl pyrazolyl, C₁-C₄ alkyl substituted pyrazinyl, C₁-C₄ alkyl substituted isoxazolyl, benzoisoxazolyl, morpholinomethyl, methylthiomethyl, methoxymethyl, N-methyl imidazolyl, and imidazolyl.

38. At least one chemical entity of claim 1, wherein R₃ is —C(O)R₇ and R₇ is optionally substituted phenyl.

39. At least one chemical entity of claim 38, wherein R₃ is —C(O)R₇ and R₇ is tolyl, halophenyl, methylhalophenyl, hydroxymethylphenyl, halo(trifluoromethyl)phenyl-, methylenedioxyphenyl, formylphenyl or cyanophenyl.

40. At least one chemical entity of claim 1, wherein R₃ is SO₂R₇ₐ and R₇ₐ is chosen from C₁-C₁₃ alkyl; phenyl; naphthyl; phenyl substituted with halo, C₁-C₄ alkyl, C₁-C₄ alkoxy, cyano, nitro, methylenedioxy, or trifluoromethyl; biphenylyl; and heteroaryl.

41. At least one chemical entity of claim 1 wherein
one of T and T′ is absent and the other is optionally substituted alkylene;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-;
R_{2′} is hydrogen;
R₄ is hydrogen, cyano, or optionally substituted methyl;
R₅ is hydrogen, methyl, or cyano; and
R₃ taken together with R₆ and the nitrogen to which they are bound, form an optionally substituted imidazolyl ring.

42. At least one chemical entity of claim 1 wherein
T and T′ are independently optionally substituted alkylene;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-;
R_{2′} is hydrogen;
R₄ is hydrogen, cyano, or optionally substituted methyl;
R₅ is hydrogen, methyl, or cyano; and
R₃ taken together with R₆ and the nitrogen to which they are bound, form an optionally substituted imidazolyl ring.

43. At least one chemical entity of claim 1 wherein
one of T and T′ is absent and the other is optionally substituted alkylene;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-;
R_{2′} is hydrogen;
R₄is hydrogen, cyano, or optionally substituted methyl;
R₅ is hydrogen, methyl, or cyano; and
R₃ taken together with R₆ form an optionally substituted imidazolinyl ring.

44. At least one chemical entity of claim 1 wherein
T and T′ are independently optionally substituted alkylene;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-;
R_{2′} is hydrogen;
R₄ is hydrogen, cyano, or optionally substituted methyl;
R₅ is hydrogen, methyl, or cyano; and
R₃ taken together with R₆ form an optionally substituted imidazolinyl ring.

45. At least one chemical entity of claim 1 wherein
T and T′ are independently optionally substituted alkylene or absent;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-;
R_{2′} is hydrogen;
R₄ is hydrogen, cyano, or optionally substituted methyl;
R₅ is hydrogen, methyl, or cyano; and
R₃ taken together with R₆ form an optionally substituted piperazine- or diazepane ring.

46. At least one chemical entity of claim 1 wherein
T and T′ are independently optionally substituted alkylene or absent;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl;
R_{2′} is hydrogen;
R₄ is hydrogen, cyano, or optionally substituted methyl;
R₅ is hydrogen, methyl, or cyano; and
R₃ taken together with R₆ form an optionally substituted diazepinone ring.

47. At least one chemical entity of claim 1 wherein
one of T and T′ is absent and the other is optionally substituted alkylene;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-,
R_{2′} is hydrogen;
R₄ is hydrogen, cyano, or optionally substituted methyl;
R₅ is hydrogen, methyl, or cyano;
R₆ is R₁₆-alkylene-;
R₁₆ is amino, C₁-C₄ alkylamino-, di(C₁-C₄ alkyl)amino-, C₁-C₄ alkoxy-, hydroxy, or N-heterocyclyl;
R₃ is —C(O)R₇; and
R₇ is optionally substituted phenyl.

48. At least one chemical entity of claim 1 wherein
T and T′ are independently optionally substituted alkylene;
R₁ is benzyl, halobenzyl, methylbenzyl, hydroxybenzyl, cyanobenzyl, methoxybenzyl, or naphthalenylmethyl;
R₂ is optionally substituted C₁-C₄ alkyl-,
R_{2′} is hydrogen;
R₄ is hydrogen, cyano, or optionally substituted methyl;
R₅ is hydrogen, methyl, or cyano;
R₆ is R₁₆-alkylene-;
R₁₆ is amino, C₁-C₄ alkylamino-, di(C₁-C₄ alkyl)amino-, C₁-C₄ alkoxy-, hydroxy, or N-heterocyclyl;
R₃ is —C(O)R₇; and
R₇ is optionally substituted phenyl.

49. At least one chemical entity of claim 1 wherein
T and T′ are independently optionally lower alkylene or absent;
R₁ is benzyl, halobenzyl, methylbenzyl, hydroxybenzyl, cyanobenzyl, methoxybenzyl, or naphthalenylmethyl;
R₂ is hydrogen;
R₄ is hydrogen, cyano, or optionally substituted methyl;
R₅ is hydrogen, methyl, or cyano;
R₆ is R₁₆-alkylene-;
R₁₆ is amino, C₁-C₄ alkylamino-, di(C₁-C₄ alkyl)amino-, C₁-C₄ alkoxy-, hydroxy, or N-heterocyclyl; and
R₆ taken together with R₂ form an optionally substituted 5- to 12-membered nitrogen-containing heterocycle, which optionally incorporates from one to two additional heteroatoms, selected from N, O, and S in the heterocycle ring.

50. At least one chemical entity of claim 1 that is
5,6-dimethyl-2-{1-[2-(4-methylphenyl)-1-piperazinyl]propyl}-3-(phenylmethyl)-4(3H)-pyrimidinone; or
5-methyl-2-[2-methyl-1-(7-oxohexahydro-1H-1,4-diazepin-1-yl)propyl]-6-oxo-1-(phenylmethyl)-1,6-dihydro-4-pyrimidinecarbonitrile.

51. A compound according to claim 1, wherein R₂ and R_{2′} are each attached to a stereogenic center having an R-configuration, or a pharmaceutically acceptable salt or solvate thereof.

52. A pharmaceutical composition comprising a pharmaceutical excipient and at least one chemical entity of claim 1.

53. A pharmaceutical composition according to claim 52, wherein said composition further comprises a chemotherapeutic agent.

54. A pharmaceutical composition according to claim 53 wherein said chemotherapeutic agent is chosen from a taxane.

55. A pharmaceutical composition according to claim 53, wherein said chemotherapeutic agent is chosen from a vinca alkaloid.

56. A pharmaceutical composition according to claim 53, wherein said chemotherapeutic agent is chosen from a topoisomerase I inhibitor.

57. A method of inhibiting KSP which comprises contacting KSP with an effective amount of at least one chemical entity according to any one of claim 1.

58. A method for the treatment of a cellular proliferative disease comprising administering to a subject in need thereof at least one chemical entity according to claim 1.

59. A method according to claim 58 wherein said disease is selected from cancer, hyperplasias, restenosis, cardiac hypertrophy, immune disorders, and inflammation.

60. (canceled)

61. (canceled)
